(19) United States
(12) Patent Application Publication
(10) Pub. No.: US 2024/0173248 A1
(43) Pub. Date: May 30, 2024

(54) HAIR CARE COMPOSITIONS AND METHODS OF USE

(71) Applicant: Amyris, Inc., Emeryville, CA (US)

(72) Inventors: Ramya Viswanathan, Emeryville, CA (US); Imee Lin, Emeryville, CA (US); Hana Sheikh, Emeryville, CA (US)

(21) Appl. No.: 18/505,599

(22) Filed: Nov. 9, 2023

Related U.S. Application Data
(60) Provisional application No. 63/424,491, filed on Nov. 11, 2022.

(51) Int. Cl. A61K 8/9789; A61K 8/31; A61K 8/34; A61K 8/35; A61K 8/37; A61K 8/67; A61K 8/9794; A61Q 5/12; A61Q 17/04

(52) U.S. Cl. CPC A61K 8/9789; A61K 8/31; A61K 8/34; A61K 8/342; A61K 8/35; A61K 8/37; A61K 8/678; A61K 8/9794; A61Q 5/12; A61Q 17/04; A61K 2800/34

(57) ABSTRACT

Provided herein are hair care compositions containing a sugar cane derived alkanes or squalane and which are generally free from silicones. The hair care compositions provided have beneficial properties including UV and thermal protection as well as restorative and protective properties. Also provided are methods of restoring and protecting hair from damage caused by frequent styling.

### BACKGROUND

Repeated hair styling treatments and environmental factors damage hair over time. Existing solutions typically offer a temporary benefit and rely on ingredients with negative environmental impact such as volatile silicones. The hair treatments based on sugar cane derivatives including C13-15 alkanes, and squalane disclosed herein offer immediate reparative benefits as well as continuous protection during styling treatments and UV exposure.

### BRIEF SUMMARY

In one aspect the disclosure relates to a pre-wash scalp oil composition comprising a C13-15 alkane, squalane, at least one conditioning agent and at least one antioxidant. Some embodiments are directed to methods of reducing hair breakage, hair damage, hair fall, or a combination thereof, comprising administering the pre-wash scalp oil composition disclosed herein to the scalp.

Some embodiments are directed to a complete air-dry cream composition comprising a C13-15 alkane, squalane, a humectant, a film former, and at least one conditioning agent. Some embodiments are directed to methods of enhancing hair definition, reducing frizz, reducing the appearance of split ends, or any combination thereof, comprising applying the complete air-dry cream composition disclosed herein to the hair.

Some embodiments are directed to a complete nourishing shine drop composition comprising a C13-15 alkane, squalane, at least one conditioning agent, at least one emollient, and at least one antioxidant. Some embodiments are directed to methods of reducing frizz, providing frizz control, or a combination thereof, comprising applying the complete nourishing shine drop composition of disclosed to the hair.

Some embodiments are directed to a complete instant recovery serum composition comprising a C13-15 alkane, squalane, a humectant, and at least one conditioning agent. Some embodiments are directed to methods of minimizing split ends, improving hair luster, or a combination thereof, comprising applying the complete instant recovery serum composition disclosed herein to the hair.

Also disclosed are methods of protecting against thermal damage, UV damage, or a combination thereof, comprising applying complete instant recovery serum composition disclosed herein to the hair.

### BRIEF DESCRIPTION OF THE DRAWINGS

FIG. 1 is a graph showing harmonic oscillations with different ranges of motion.

FIG. 2 is a graph showing the effect of damping on harmonic oscillations.

FIG. 3 shows a hair sample (left hand side), body (middle left), body+frizz (middle right); and total area (right hand side).

FIG. 4 shows the profile of light intensity and acquisition of the diffuse reflection curves (D_Stamm) and specular reflection (S_Gaussian), as well as maximum intensity (IMAX, red circle) and total width of the Gaussian profile (w1/2, pink arrow).

FIG. 5 is a graph showing a comparison in frizz resulting from the use of a complete air-dry cream composition (093002-10) compared to a non-conditioning shampoo (CTR) at 0 and 24 hours.

FIG. 6 is a graph showing a comparison in curls resulting from the use of a complete air-dry cream composition (093002-10) compared to a non-conditioning shampoo (CTR) at 0 and 24 hours.

FIG. 7 is a graph showing a comparison in damping resulting from the use of a complete air-dry cream composition (093002-10) compared to a non-conditioning shampoo (CTR).

FIG. 8 is a graph showing a comparison in mean frizz resulting from the use of a complete air-dry cream composition (093002-10) compared to a non-conditioning shampoo (CTR) at 0 and 24 hours.

FIG. 9 is a graph showing a comparison in mean volume resulting from the use of a complete air-dry cream composition (093002-10) compared to a non-conditioning shampoo (CTR) at 0 and 24 hours.

FIG. 10 is a graph showing a comparison in split ends from the use of a complete air-dry cream composition (093002-10) compared to a non-conditioning shampoo control (CTR).

FIG. 11 is a graph showing a comparison in mean enthalpy from the use of a complete instant recovery serum composition (093002-09) compared to a non-conditioning shampoo negative control (CTRN) and non-conditioning shampoo positive control (CTRP).

FIG. 12 is a graph showing a comparison in mean luster from the use of a complete instant recovery serum composition (093002-09) compared to a non-conditioning shampoo negative control (CTRN) and non-conditioning shampoo positive control (CTRP).

FIG. 13 is a graph showing a comparison in split ends from the use of a complete instant recovery serum composition (093002-09) compared to a non-conditioning shampoo control (CTR).

FIG. 14 is a graph Results showing a comparison in color retention resulting from the use of a complete instant recovery serum composition (093002-09) compared to a non-conditioning shampoo control (CTR) after an increasing number of wash cycles (0, 5, 10, 15, 20).

FIG. 15 is a graph showing xenon lamp spectra (1 hour of exposition corresponds to 9 hours of sunlight).

FIG. 16 is a graph showing the photoluminescence spectra of tryptophan amino acid for the evaluated groups (light intensity versus wavelength).

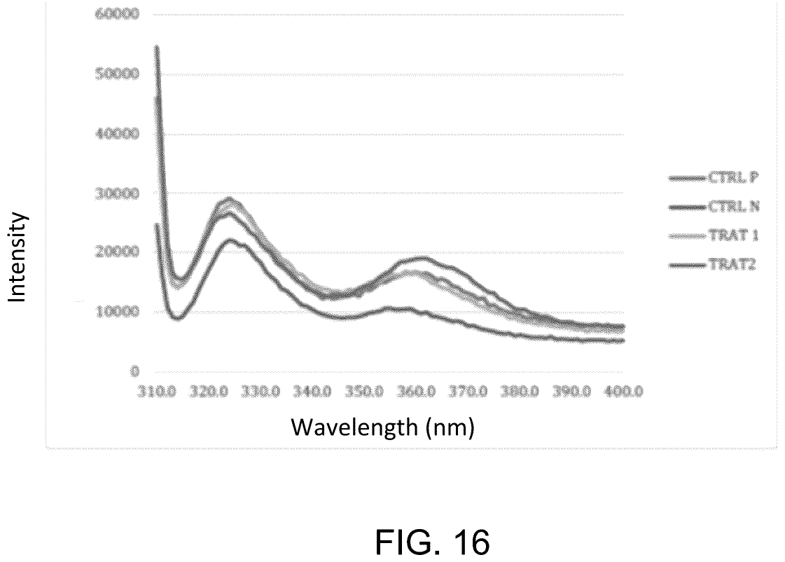

FIG. 17 is an image of the experimental automatic combing machine BLPA 101.

FIG. 18 is an image of the preparation of the hair fibers for the Tensile test.

FIG. 19 is a graph showing load versus extension for each fiber analyzed using the Instron.

FIG. 20 is a graph showing a comparison in hair breakage following treatment with a complete pre-wash scalp oil composition (093002-08) and a non-conditioning shampoo (CTR).

FIG. 21 is a graph showing a comparison in damaged areas following treatment with a complete pre-wash scalp oil composition (093002-08) and a non-conditioning shampoo (CTR).

FIG. 22 is a graph showing a comparison in split ends following treatment with a complete pre-wash scalp oil composition (093002-08) and a non-conditioning shampoo (CTR).

FIG. 23 is a graph showing a comparison in luster following treatment with a complete pre-wash scalp oil composition (093002-08) and a non-conditioning shampoo negative control (CTRN) and non-conditioning shampoo positive control (CTRP).

FIG. 24 is a comparison in mean frizz resulting from the use of a pre-wash scalp oil composition (093002-08) compared to a non-conditioning shampoo (CTR) at 0 and 24 hours.

FIG. 25. is a graph showing heat flow versus temperature profile showing the peak of Keratin denaturation.

FIG. 26 is a graph showing a comparison in mean enthalpy from the use of a nourishing shine drop composition (097009-01) compared to a non-conditioning shampoo (CTR).

FIG. 27 is a graph showing a comparison in mean damaged area from the use of a nourishing shine drop composition (097009-01) compared to a non-conditioning shampoo (CTR).

FIG. 28 shows a humidity and temperature control chamber (left) and images acquisition system.

FIG. 29 shows a graph a comparison in frizz from the use of a nourishing shine drop composition (097009-01) compared to a non-conditioning shampoo (CTR) at 0 hours.

FIG. 30 shows a comparison in frizz from the use of a nourishing shine drop composition (097009-01) compared to a non-conditioning shampoo (CTR) at 24 hours.

FIG. 31 illustrates the degree of frizz in hair samples treated with a non-conditioning shampoo (CTR) at 0 hours and 24 hours.

FIG. 32 illustrates the degree of frizz in hair samples treated with a nourishing shine drop composition (097009-01) at 0 hours and 24 hours.

FIG. 33 is a comparison in mean volume resulting from the use of a nourishing shine drop composition (097009-01) compared to a non-conditioning shampoo (CTR) at 24 hours.

FIG. 34 illustrates the degree of volume in hair samples treated with a non-conditioning shampoo (CTR) at 0 hours and 24 hours.

FIG. 35 illustrates the degree of volume in hair samples treated with a nourishing shine drop composition (097009-01) at 0 hours and 24 hours.

FIG. 36 is a graph showing the photoluminescence spectra of tryptophan amino acid for the evaluated groups (light intensity versus wavelength).

### DETAILED DESCRIPTION

In the following description, numerous specific details are set forth in order to provide a thorough understanding of the present embodiments. It will be apparent, however, to one having ordinary skill in the art that the specific detail need not be employed to practice the present embodiments. In other instances, well-known materials or methods have not been described in detail in order to avoid obscuring the present embodiments.

Reference throughout this specification to “one embodiment”, “an embodiment”, “one example” or “an example” means that a particular feature, structure or characteristic described in connection with the embodiment or example is included in at least one embodiment of the present embodiments. Thus, appearances of the phrases “in one embodiment”, “in an embodiment”, “one example” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

As used herein, the terms “comprises,” “comprising,” “includes,” “including,” “has,” “having,” or any other variation thereof, are intended to cover a non-exclusive inclusion. For example, a process, article, or apparatus that comprises a list of elements is not necessarily limited to only those elements but may include other elements not expressly listed or inherent to such process, article, or apparatus.

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Additionally, any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” and “in one embodiment.”

The hair care compositions disclosed herein rely on sugar cane derived C13-15 alkanes, squalane or combinations thereof as the active agent within the compositions.

As used herein, “hemisqualane”, which is also referred to as “farnesane”, refers to a compound having the following formula:

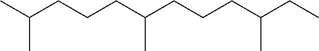

As used herein, “squalane” refers to a compound having the following formula:

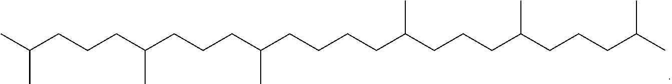 [SMILES: CC(C)CCCC(C)CCCC(C)CCCCC(C)CCCC(C)CCCC(C)C]

In some embodiments, the C13-15 alkane is hemi-squalane. In some embodiments, the C13-15 alkane is an unsubstituted alkane. The compositions of the invention include squalane. Squalane acts as an emollient in the compositions disclosed herein and replaces volatile silicone-based ingredients such as isohexadecane, amodimethicone, and cyclomethicones like cyclopentasiloxane (D5).

The hair care compositions disclosed herein may also contain one or more solvents. Solvents are used to dissolve various solutes that have one or more functions in the formulation. Solutes dissolved by the solvent may function as chelating agents, surfactants, humectants, emulsion stabilizers, viscosity increasing agents, emulsifying agent, additional emollients in addition to squalane, dispersing agents, conditioning agents, preservatives, conditioning agents, and aromatic agents. A preferred solvent of the invention is water.

The compositions disclosed herein may contain one or more chelating agents. Chelating agents are chemical compounds that react with metal ions to form stable, water-soluble complexes. Illustrative chelating agents include sodium gluconate, sodium phytate, EDTA, tetrasodium glutamate diacetate, trisodium ethylene diamine disuccinate. A preferred chelating agent of the invention is sodium gluconate.

The compositions disclosed herein may contain one or more surfactants. Surfactants function to lower the surface tension of one or more liquids of the formulation. Illustrative surfactants include caprylyl/capryl glucoside, coco-glucoside, isostearic acid, cetearyl glucoside, and arachidyl glucoside. Preferred surfactants useful in the formulation include sorbitan oleate decylglucoside crosspolymer caprylyl/capryl glucoside, coco-glucoside, and isostearic acid.

The compositions disclosed herein may also contain one or more humectants. Humectants are compounds that retain the moisture of the formulation and are typically hygroscopic compounds having multiple hydrophilic groups. Illustrative humectants include glycerin, propanediol, propylene glycol, hexylene glycol, butylene glycol, sorbitol, and xylitol. A preferred humectant of the formulation is glycerin.

The compositions disclosed herein may include one or more viscosity increasing agents. Viscosity increasing agents are compounds that act by thickening the formulation and thereby increasing the overall viscosity of the sunscreen formulation. Illustrative viscosity increasing agents include cetyl palmitate, cetearyl alcohol, methyl dihydroabietate, behenyl alcohol, brassica alcohol, arachidyl alcohol, coconut alcohol, sorbitan palmitate. Preferred viscosity increasing agents include acrylates/c10-30 alkyl acrylate crosspolymers, sodium chloride, cetyl palmitate, cetearyl alcohol, and methyl dihydroabietate.

The compositions disclosed herein may include one or more emulsifying agents or emulsifier. Emulsifying agents are compounds that keep dissimilar chemicals (such as hydrophobic and hydrophilic compounds) from separating in an emulsion. Illustrative emulsifying agents include sorbitan olivate, polyglyceryl-3 polyricinoleate, lecithin, glyceryl stearate, cetearyl olivate. Preferred emulsifying agents include sorbitan olivate, polyglyceryl-3 polyricinoleate, and lecithin.

The compositions disclosed herein may contain one or more conditioning agents. Suitable conditioning agents include oryza sativa (rice) bran extract, helianthus annuus (sunflower) extract, bisabolol, ocimum basilicum hairy root culture extract, helianthus annuus (sunflower) seed oil, cocos nucifera (coconut) oil, curcuma longa root extract, melia azadirachta flower extract, melia azadirachta leaf extract, melia azadirachta bark extract, ocimum sanctum leaf extract, corallina officinalis extract, ocimum basilicum flower/leaf extract, eclipta prostrata extract, melia azadirachta extract, moringa oleifera seed oil, hydrolyzed jojoba esters, linum usitatissimum (linseed) seed extract, salvia hispanica seed extract, caprylic acid, xylitol, cinnamidopropyltrimonium chloride, ethylhexylglycerin and any combination thereof and any combination thereof.

The compositions disclosed herein may contain one or more antioxidants. Illustrative antioxidants include rosmarinus officinalis (rosemary) leaf extract, tocopherol, and a combination thereof.

The compositions disclosed herein may contain one or more film forming agent. In some embodiments, the film forming agent is a maltodextrin/vinyl pyrrolidone copolymer.

The compositions disclosed herein may contain one or more preservatives. Illustrative preservatives include phenoxyethanol, benzyl alcohol, hydroxyacetophenone, chlorophensin, potassium sorbate, and ethylhexylglycerin.

The compositions disclosed herein may contain an aromatic component. In some embodiments, the aromatic component includes a fragrance, citral, limonene, geraniol, linalool, citrus reticulata (tangerine) peel oil, citral, linalool, limonene, jasminum officinale (jasmine) oil, benzyl benzoate, linalool, and any combination thereof.

As used herein an “effective amount” means an amount necessary to at least partly attain the desired response, or to delay the onset or inhibit progression or halt altogether, the onset or progression of a particular symptom being treated. The amount varies depending upon the health and physical condition of the subject to be treated, the taxonomic group of subject to be treated, the degree of protection desired, the formulation of the composition, the assessment of the medical situation, and other relevant factors. It is expected that the amount will fall in a relatively broad range that can be determined through routine trials.

As used herein, “subject” or “patient” is an organism that is treated using one of the methods of the present disclosure. In an embodiment, the subject is a mammalian subject, such as a human or a domestic animal.

As used herein, the term “about” will be understood by persons of ordinary skill in the art and will vary to some extent on the context in which is used. If there are uses of the term which are not clear to persons of ordinary skill in the art given the context in which is used, “about” may mean up to plus or minus 10% of the particular term.

### Pre-Wash Scalp Oil Composition and Methods of Use

Embodiments herein are directed to pre-wash scalp oil compositions comprising a C13-15 alkane, squalane, at least one conditioning agent and at least one antioxidant. In some embodiments, the C13-15 alkane is hemisqualane. In some embodiments, the squalane comprises between about 80% w/w and about 99% w/w of the composition. In some embodiments, the squalane comprises about 91% w/w of the composition. In some embodiments, the C13-15 alkane comprises between about 1% w/w and about 10% w/w of the composition. In some embodiments, the C13-15 alkane comprises about 5% w/w of the composition.

In some embodiments, the conditioning agent is selected from the group consisting oryza sativa (rice) bran extract, helianthus annuus (sunflower) extract, bisabolol, ocimum basilicum hairy root culture extract, helianthus annuus (sunflower) seed oil, cocos nucifera (coconut) oil, curcuma longa root extract, melia azadirachta flower extract, melia azadirachta leaf extract, melia azadirachta bark extract, ocimum sanctum leaf extract, corallina officinalis extract, ocimum basilicum flower/leaf extract, eclipta prostrata extract, melia azadirachta extract, moringa oleifera seed oil, and any combination thereof.

In some embodiments, the antioxidant is selected from the group consisting of rosmarinus officinalis (rosemary) leaf extract, tocopherol, and a combination thereof.

Some embodiments further comprising an aromatic component. In some embodiments, the aromatic component is selected from the group consisting of fragrance, citral, limonene, geraniol, linalool, citrus reticulata (tangerine) peel oil, citral, linalool, limonene, jasminum officinale (jasmine) oil, benzyl benzoate, linalool, and any combination thereof.

In some embodiments, the composition is substantially free from silicones. In some embodiments, the composition is free from silicones.

Some embodiments are directed to methods for reducing hair breakage, hair damage, hair fall, or a combination thereof, comprising administering the pre-wash scalp oil composition disclosed herein to the scalp. In some embodiments, the methods further comprise massaging the pre-wash scalp oil composition disclosed herein into the scalp, leaving on for about 10 to about 15 minutes, followed by washing scalp and hair with shampoo.

Some embodiments are directed to methods for reducing hair breakage, hair damage, hair fall, or a combination thereof, comprising administering an effective amount the pre-wash scalp oil composition disclosed herein to the scalp. In some embodiments, the methods further comprise massaging the pre-wash scalp oil composition disclosed herein into the scalp, leaving on for about 10 to about 15 minutes, followed by washing scalp and hair with shampoo.

### Complete Air-Dry Cream Compositions and Methods of Use

Embodiments herein are directed to a complete air-dry cream composition comprising a C13-15 alkane, squalane, a humectant, a film former, and at least one conditioning agent. In some embodiments, the C13-C15 alkane is hemisqualane.

In some embodiments, the humectant is glycerin. In some embodiments, the film former is a maltodextrin/vinyl pyrrolidone copolymer. In some embodiments, the at least one conditioning agent is selected from the group consisting of hydrolyzed jojoba esters, linum usitatissimum (linseed) seed extract, salvia hispanica seed extract, caprylic acid, xylitol, cinnamidopropyltrimonium chloride, ethylhexylglycerin and any combination thereof. In some embodiments, the at least one conditioning agent is linum usitatissimum (linseed) seed extract, salvia hispanica seed extract or a combination thereof.

In some embodiments, the C13-15 alkane comprises between about 1% w/w and about 10% w/w of the composition. In some embodiments, the C13-15 alkane comprises about 5% w/w of the composition. In some embodiments, the squalane comprises between about 0.1% w/w and about 1.0% w/w of the composition. In some embodiments, the squalane comprises about 0.5% w/w of the composition. In some embodiments, the glycerin comprises between about 0.1% w/w and 10% w/w of the composition. In some embodiments, the glycerin comprises between about 2% w/w of the composition. In some embodiments, the maltodextrin/vinyl pyrrolidone copolymer comprises between about 0.1% w/w and 1.0% w/w of the composition. In some embodiments, the maltodextrin/vinyl pyrrolidone copolymer comprises between about 0.6% w/w of the composition. In some embodiments, the linseed extract comprises between about 0.1% w/w and 1.0% of the composition. In some embodiments, the linseed extract comprises about 0.35% w/w of the composition. In some embodiments, the salvia hispanica seed extract comprises between about 0.1% w/w and 1.0% of the composition. In some embodiments, the salvia hispanica seed extract comprises about 0.2% w/w of the composition.

Some embodiments further comprise at least one of a solvent, a viscosity increasing agent, a chelator, a preservative, a pH adjustor, a hair fixative, a preservative, an antioxidant, an emollient, a surfactant, or an aromatic component.

In some embodiments, the solvent is water.

In some embodiments, the viscosity increasing agent is selected from the group consisting of an acrylate/C10-30 alkyl acrylate crosspolymer, sodium chloride and any combination thereof.

In some embodiments, the chelator is sodium gluconate.

In some embodiments, the preservative is selected from the group consisting of potassium sorbate, phenoxyethanol, ethylhexylglycerin and benzyl alcohol.

In some embodiments, the pH adjustor is sodium hydroxide.

In some embodiments, the hair fixative is polyvinylpyrrolidone.

In some embodiments, the preservative is selected from potassium sorbate, phenoxyethanol, ethylhexylglycerin, and benzyl alcohol and any combination thereof.

In some embodiments, the antioxidant is tocopherol.

In some embodiments, the emollient is selected from moringa oleifera seed oil, butyrospermum parkii (shea) butter, and any combination thereof.

In some embodiments, the surfactant is sorbitan oleate decylglucoside crosspolymer.

In some embodiments, the aromatic component is selected from fragrance, amyl cinnamic alcohol, citronellol, coumarin, limonene, eugenol, hexyl cinnamic aldehyde, hydroxycitronellal, linalool, methyl ionone gamma and any combination thereof.

In some embodiments, the composition is substantially free from silicones. In some embodiments, the composition is free from silicones.

Some embodiments are directed to methods of enhancing hair definition, reducing frizz, reducing the appearance of split ends, or any combination thereof, comprising applying the complete air-dry cream composition disclosed herein to the hair. In some embodiments, the hair is damp or wet prior to applying the composition.

Some embodiments are directed to methods of enhancing hair definition, reducing frizz, reducing the appearance of split ends, or any combination thereof, comprising applying an effective amount of the complete air-dry cream composition disclosed herein to the hair. In some embodiments, the hair is damp or wet prior to applying the composition.

### Complete Nourishing Shine Drop Composition and Methods of Use

Embodiments herein are directed to complete nourishing shine drop compositions comprising a C13-15 alkane, squalane, at least one conditioning agent, at least one emollient, and at least one antioxidant.

In some embodiments, the C13-15 alkane is hemisqualane.

In some embodiments, the at least one conditioning agent is selected from the group consisting of helianthus annuus (sunflower) seed oil, hibiscus sabdariffa flower extract, eclipata prostrata extract, melia azadirachta extract and any combination thereof.

In some embodiments, the at least one conditioning agent is hibiscus sabdariffa flower extract.

In some embodiments, the at least one emollient is selected from the group consisting of jojoba esters, meadowfoam estolide, limnanthes alba (meadowfoam) seed oil, moringa oleifera seed oil, and any combination thereof.

In some embodiments, the at least one emollient is meadowfoam estolide.

In some embodiments, the antioxidant is selected from rosmarinus officinalis (rosemary) leaf extract, tocopherol, and any combination thereof.

Some embodiments further comprise at least one aromatic component. In some the at least one aromatic component is selected from the group consisting of a fragrance, amyl cinnamal, citronellol, coumarin, limonene, eugenol, hexyl cinnamal, hydroxycitronellal, linalool, methyl ionones, and any combination thereof.

In some embodiments, the composition is substantially free from silicones. In some embodiments, the composition is free from silicones.

In some embodiments, the C13-15 alkane comprises between about 40% w/w and about 80% w/w of the composition. In some embodiments, the C13-15 alkane comprises about 60% w/w of the composition. In some embodiments, the squalane comprises between about 1.0% w/w and about 30% w/w of the composition. In some embodiments, the squalane comprises about 20% w/w of the composition. In some embodiments, the meadowfoam estolide comprises between about 0.1% w/w and about 10% w/w of the composition. In some embodiments, the meadowfoam estolide comprises about 2% w/w of the composition. In some embodiments, the hibiscus sabdariffa flower extract comprises between about 0.01% w/w to about 0.1% w/w of the composition. In some embodiments, the hibiscus sabdariffa flower extract comprises about 0.06% w/w of the composition. In some embodiments, the hibiscus sabdariffa flower extract comprises about 0.06% w/w of the composition. In some embodiments, the antioxidant comprises between about 0.0001% w/w and 1% w/w of the composition.

Some embodiments are directed to methods of reducing frizz, providing frizz control, or a combination thereof, comprising applying the complete nourishing shine drop composition of disclosed herein to the hair. Some embodiments are directed to methods of reducing frizz, providing frizz control, or a combination thereof, comprising applying an effective amount of the complete nourishing shine drop composition disclosed herein to the hair.

### Complete Instant Recovery Serum and Methods of Use

Embodiments herein are directed to complete instant recovery serum compositions comprising a C13-15 alkane, squalane, a humectant, and at least one conditioning agent.

In some embodiments, the C13-C15 alkane is hemisqualane.

In some embodiments, the humectant is glycerin.

In some embodiments, the at least one conditioning agent is selected from niacinamide, hydroxypropyltrimonium lemon protein, leuconostoc/radish root ferment filtrate, ethylhexylglycerin, tocopherol and any combination thereof.

Some embodiments further comprise a solvent, a chelator, a stabilizer, an emulsifier, a preservative, a pH adjustor, at least one aromatic component, or any combination thereof.

In some embodiments, the solvent is water.

In some embodiments, the chelator is sodium gluconate.

In some embodiments, the stabilizer is a sodium acrylate copolymer.

In some embodiments, the emulsifier is lecithin.

In some embodiments, the preservative is benzyl alcohol.

In some embodiments, the pH adjustor is citric acid.

In some embodiments, the aromatic component is selected from the group consisting of fragrance, citral, citronellol, coumarin, limonene, geraniol, linalool, citrus reticulata (tangerine) peel oil, citral, citronellol, geraniol, linalool, limonene, jasminum officinale (jasmine) oil, benzyl alcohol, benzyl benzoate, eugenol, linalool, and any combination thereof.

In some embodiments, the composition is substantially free from silicones. In some embodiments, the composition is free from silicones.

In some embodiments, the C13-15 alkane comprises between about 1% w/w and about 10% w/w of the composition. In some embodiments, the C13-15 alkane comprises about 5% w/w of the composition. In some embodiments, the squalane comprises between about 0.1% w/w and about 10% w/w of the composition. In some embodiments, the squalane comprises about 1% w/w of the composition. In some embodiments, the humectant comprises between about 1% w/w and 10% w/w of the composition. In some embodiments, the humectant comprises about 3% w/w of the composition. In some embodiments, the at least one conditioning agent comprises between about 0.0001% w/w and 10% w/w of the composition. In some embodiments, the at least one conditioning agent comprises about 0.5% w/w of the composition.

Some embodiments are directed to methods of minimizing split ends, improving hair luster, or a combination thereof, comprising applying the complete instant recovery serum compositions disclosed herein to the hair. In some embodiments, the hair is damp.

Some embodiments are directed to methods of minimizing split ends, improving hair luster, or a combination thereof, comprising applying an effective amount of the complete instant recovery serum compositions disclosed herein to the hair. In some embodiments, the hair is damp.

Some embodiments are directed to methods of protecting against thermal damage, UV damage, or a combination thereof, comprising applying the complete instant recovery serum compositions disclosed herein to the hair. In some embodiments, the hair is damp.

Some embodiments are directed to methods of protecting against thermal damage, UV damage, or a combination thereof, comprising applying an effective amount of the complete instant recovery serum compositions disclosed herein to the hair. In some embodiments, the hair is damp.

### EXAMPLES

The following examples illustrate the properties of the compositions disclosed herein.

### Example 1: Evaluation of a Complete Air-Dry Cream Composition

Hair is a natural keratin fiber, a protein composed of a high degree of sulfur content present in the cysteine compounds. The mechanical properties observed in the hair depend, mainly, on its geometric structure and are very important in the evaluation of elasticity, softness and smoothness, combability, shine and so on. The evaluation of these properties by different methodologies can provide a better understanding of the real action of products on the hair to provide better marketing communications for these products.

The aim of the present study was to evaluate curl retention, damping, frizz and volume, luster and split ends compared to a non-conditioning shampoo. In the present study, 5 methodologies were used to evaluate a complete air-dry cream compared to a conventional non-conditioning shampoo: Curl retention: to evaluate retention and control; damping: to evaluate constant measures how quickly the movement ends; frizz and volume: to evaluate the frizz and volume reduction and control; luster (shine): to evaluate shine on natural hair; and split ends: to evaluate split ends reduction.

### Study Protocols

Hair swatches (six Brazilian natural hair swatches type V of 30 cm and 3 grams) were prepared and separated into two different groups as follows: 1.) Control (swatches washed with a non-conditioning shampoo (20% SLES)); and 2.) Treatment Group: swatches washed with a complete air-dry cream composition. Each of the groups were then treated with the non-conditioning shampoo before the start of the study to remove any residues in the hair according to the following process: 0.4 ml of non-conditioning shampoo per gram of hair was applied on the hair swatches and spread onto wet hair. The swatch was massaged 6 times from the root to the ends (three times each side of the swatch); followed by rinsing for 30 seconds with tap water (5±1 L/min; 35±2° C.). After the step of removing the waste from a separate group as a control for later evaluation. The treatment group was washed with the complete air-dry cream composition as follows: the swatches were rinsed for 30 seconds with tap water (5±1 L/min; 35±2° C.) and the excess of water was removed and 0.2 mL of complete air-dry cream composition per gram of hair was applied to the wet hair swatches and spread. The hair swatches were massaged 6 times from the root to the tips (three times each side of the swatch).

### Curl Retention

After application of the products to the hair swatches, photographs were taken (T0) and the swatches were inserted into a humidity and temperature control chamber (85±5% RH; 22±2° C.) where they remained for 24 hours. After the process of exposure to high humidity, the hair swatches were photographed again (T24). The images of each time point (T0, T24) were analyzed via ImageJ® using an internally developed macro for frizz and volume quantification. With the frizz and volume data, the Body (Volume−frizz) was also calculated.

### Damping

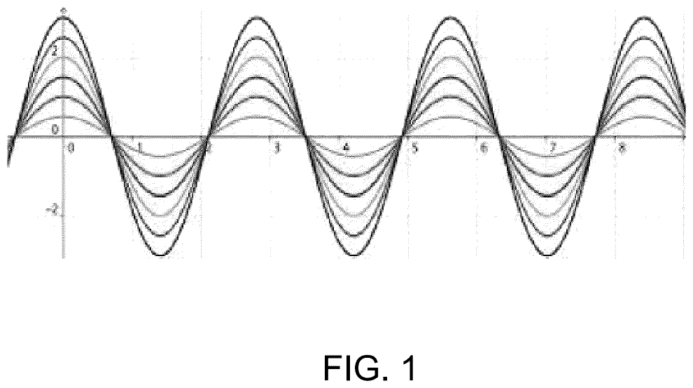

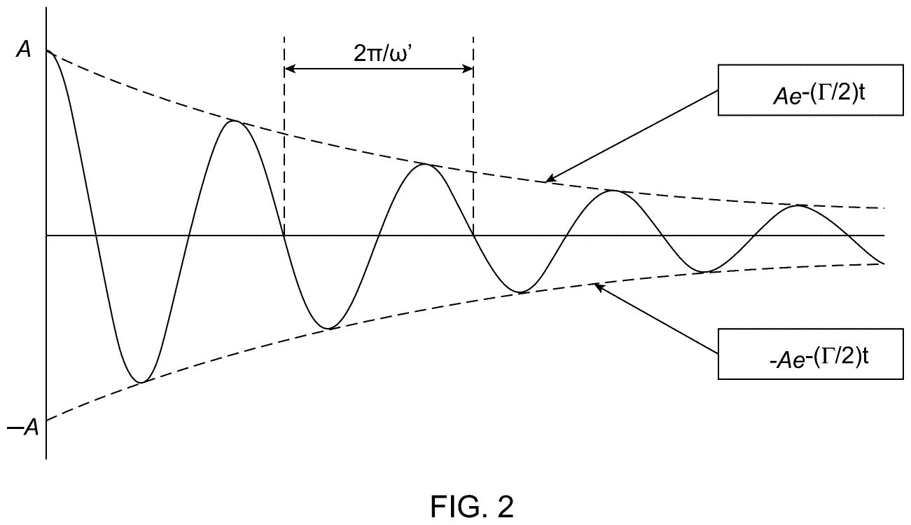

Curls are like springs and thus have an oscillating movement very similar to that of a harmonic oscillator (See FIG. 1). However, unlike the ideal oscillation in which the spring moves freely and without any external interference, real springs have so-called damping which is related to how quickly the oscillatory movement ceases. This concept is widely used in various applications from a simple car bumper to sophisticated engineering projects for bridges and buildings. FIG. 2 shows this phenomenon and shows the mathematical treatment used in these situations. Note from the FIG. 2 that the sinusoidal harmonic oscillation is involved by an exponential function in which the damping constant (Γ) represents how quickly the oscillatory motion ceases to exist. The greater the constant, the greater the damping and the lesser the movement. In the applications detailed above, the aim is precisely to cease movement as quickly as possible without the structure suffering irreversible damage on impact. The attention given in this evaluation of curly hair movement is different. The idea is to measure the damping constant and see how much it influences the movement of curly hair. If the intention is to get more movement, a higher value of the damping constant is expected, otherwise, a lower value is expected when comparing natural hair and hair after application of a treatment. In practice, the movement is not as fluid as shown in FIG. 1 and FIG. 2 due to external disturbances inherent to the system. However, it is possible with a good approximation to identify this constant and use it in the evaluations of curly hair movement.

### Frizz and Volume

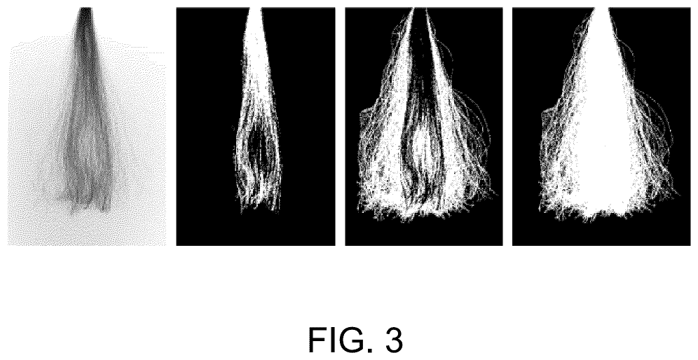

The quantification of frizz and volume of the images obtained was done with the software ImageJ® by using an internally developed macro, in which the frizz can be separated from the body of the hair sample and the area used to assess the 2D volume of the hair swatch (See FIG. 3). As mentioned, the frizz phenomena appear during the natural drying process of the hair swatches, and it becomes more emphasized in areas where the relative humidity are more elevated. Through the appropriate methodology, it is possible to identify both reduction and control of frizz and volume provided by a certain treatment. The images obtained during the study were stored and the results of frizz and volume were sent for statistical comparison as described in the following section.

### Luster

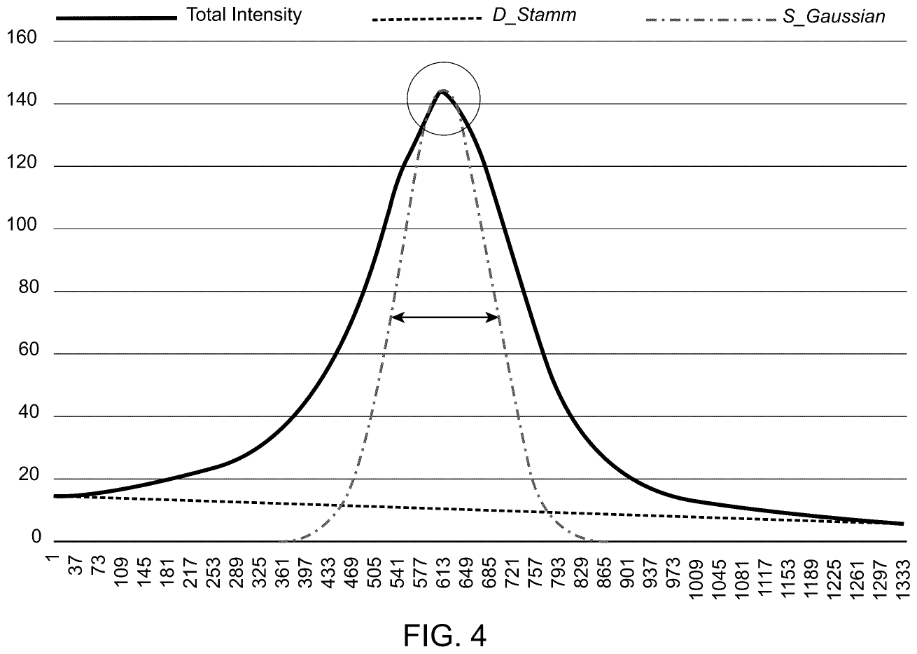

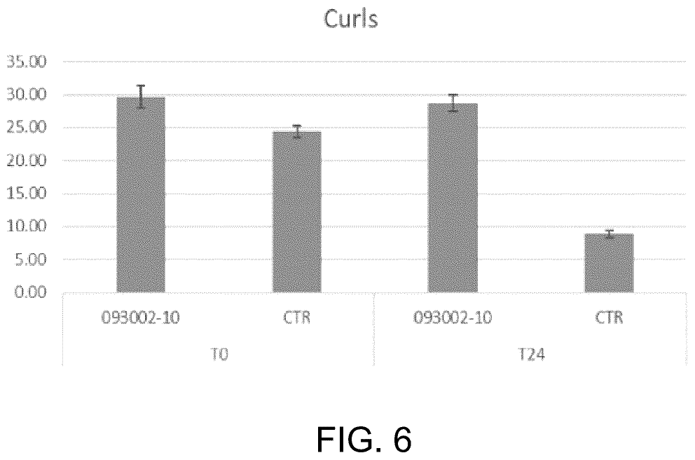

Images were photographed in a luster chamber. The images obtained are treated and analyzed with the software program ImageJ® in order to obtain the diffuse and specular light profiles of each image (See FIG. 4) to calculate luster as presented in equation below. From the diffuse reflection and specular reflection curves of the total intensity profile presented in FIG. 6, the areas under the curve of both reflections are calculated from numeric methods

$${B}_{ALL} = ( \frac{{D}_{Stamm}}{{S}_{Gaussian}} ) \times \frac{{I}_{MAX}}{{W}_{1 / 2}}$$

of approximation and used for the calculation of luster in which B_{ALL} is the value of luster defined by the adjusted equation from the equation above, DStamm is the diffuse light reflection, SGaussian is the specular light reflection, IMAX is the maximum light intensity and w1/2 is the maximum width at half-height, or the width of specular reflection shown in FIG. 4.

### Split Ends

After application of the investigational product, the hair swatches were positioned in an automatic combing machine and brushed 16000 times on their ends so as to promote the appearance of split ends, and at each cycle of 200 brushings, the split ends were counted with the aid of a magnifying glass. The value obtained was tabulated and sent for statistical analysis as described below.

### Analysis of Results

### Statistical Analysis

Exploratory data analysis was performed (mean, standard error, standard deviation, minimum, median, maximum, 95% Confidence Interval and charts). Treatments were compared through Student's t test. Software used was XLSTAT 2019 and MINITAB 14. Confidence level: 95%.

### Analysis of Results

The equations shown below can be used, although a difference is always observed, to estimate the percentage of breakage reduction and the how much less breakage compared to 20% SLES:

$$\% BR = ( 1 - \frac{TRT}{SLES} ) \times 100 \\ Number of times = {( \frac{TRT}{SLES} )}^{- 1}$$

### Results

### Curl Retention and Definition

Table 1 below summarizes the statistical results obtained for each treatment for analysis of frizz.

TABLE I
Descriptive statistics and results of
the comparison between treatments.

| | |
|---|---|
| T0 | T24 |

| Statistics | 093002-10 | CTR | 093002-10 | CTR |
|---|---|---|---|---|
| Mean | 7.70 | 10.75 | 8.52 | 20.62 |
| St. Error | 0.65 | 0.92 | 1.42 | 2.27 |
| 95% CI | [6.03 ± | [8.37 ± | [4.87 ± | [14.79 ± |
| | 9.37] | 13.13] | 12.17] | 26.45] |
| St. Dev. | 1.60 | 2.27 | 3.47 | 5.55 |
| Median | 7.96 | 11.22 | 7.06 | 19.04 |
| Minimum | 4.88 | 7.30 | 5.18 | 15.06 |
| Maximum | 9.51 | 13.78 | 14.25 | 29.44 |

| | | |
|---|---|---|
| P-Value | 0.027* | 0.002* |

*Significant at the 5% level (Student's t test)

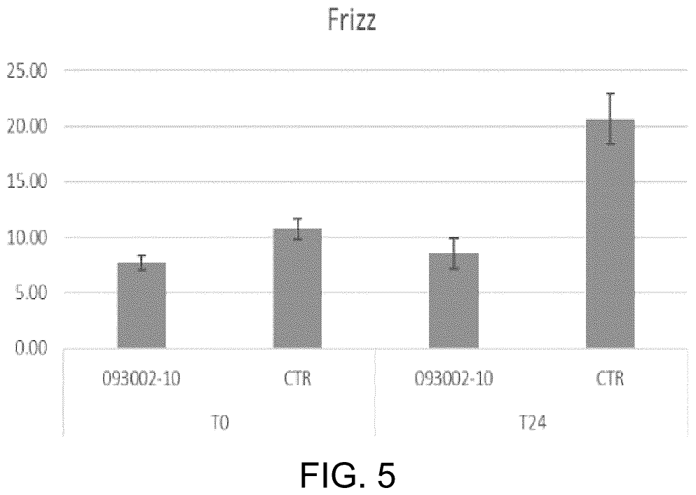

From the data presented in Table 1, FIG. 5 and the statistical analyses performed, it was possible to observed that the mean for treatment 092003-10 was significantly lower compared to control for time-points T0 and T24, and that no statistically significant difference on the mean was observed between time-points T0 and T24 for treatment 092002-10. The mean for CTR was significantly lower at time-point T0 compared to time-point T24. Table 2 summarizes the statistical results obtained for each treatment for analysis of number curls.

TABLE 2
Descriptive statistics and results of
the comparison between treatments.

| | |
|---|---|
| T0 | T24 |

| Statistics | 093002-10 | CTR | 093002-10 | CTR |
|---|---|---|---|---|
| Mean | 29.67 | 24.33 | 28.67 | 8.83 |
| St. Error | 1.69 | 0.95 | 1.26 | 0.48 |
| 95% CI | [25.33 ± | [21.88 ± | [25.44 ± | [7.6 ± |
| | 34.01] | 26.78] | 31.9] | 10.06] |
| St Dev. | 4.13 | 2.34 | 3.08 | 1.17 |
| Median | 29.50 | 24.50 | 29.50 | 9.00 |
| Minimum | 23.00 | 21.00 | 23.00 | 7.00 |
| Maximum | 34.00 | 28.00 | 32.00 | 10.00 |
| p-value | 0.028* | | <0.001* | |

*Significant at the 5% level (Student's t test)

From the data presented in Table 2, FIG. 6 and the statistical analyses performed, it was possible to observed that the mean for treatment 092003-10 was significantly higher compared to control for time-points T0 and T24. No statistically significant difference on the mean was observed between time-points T0 and T24 for treatment 092002-10. The mean for CTR was significantly lower at time-point T24 compared to time-point T0.

### Damping

Table 3 summarizes the statistical results obtained for each treatment for analysis of damping.

TABLE 3
Descriptive statistics and results
of the comparison between treatment

| | CTR | 093002-10 |
|---|---|---|
| Mean | 26.67 | 24.17 |
| St. Error | 3.33 | 1.54 |
| 95% CI | [18.1 ± 35.24] | [20.22 ± 28.12] |
| St. Dev. | 8.16 | 3.76 |
| Median | 27.50 | 25.00 |
| Minimum | 15.00 | 20.00 |
| Maximum | 35.00 | 30.00 |

| | |
|---|---|
| p-value | 0.518 |

* Significant at 5%; (Student's test)

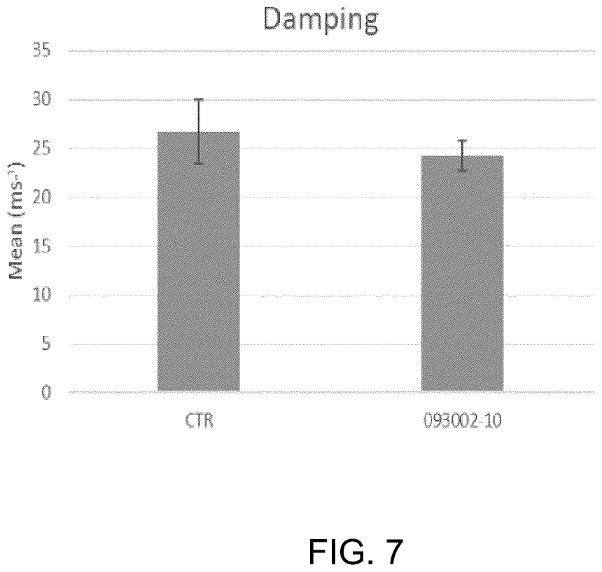

From the data presented in Table 3. FIG. 7 and the statistical analyses performed. it was possible to observed that no statistically significant difference was observed in damping for treatment 093002-10 compared to the damping for control.

### Frizz

The table 4 summarizes the statistical results obtained for each treatment for analysis of frizz.

TABLE 4
Descriptive statistics and results of
the comparison between treatments

| | T0 | | T24 | |
|---|---|---|---|---|
| Statistics | 093002-10 | CTR | 093002-10 | CTR |
| | 3.24 | 103.07 | 4.01 | 88.86 |
| | 3.04 | 97.60 | 3.17 | 118.77 |
| | 2.55 | 96.55 | 5.23 | 108.14 |
| | 3.20 | 80.78 | 4.22 | 94.31 |
| | 3.50 | 96.63 | 3.74 | 97.26 |
| | 4.49 | 118.10 | 12.06 | 111.08 |
| Mean | 3.34 | 98.79 | 5.41 | 103.07 |
| Standard | 0.26 | 4.92 | 1.36 | 4.65 |
| error | | | | |
| 95% CI | [2.66; 4.01] | [86.14; 111.44] | [1.91; 8.9] | [91.12; 115.03] |
| Standard | 0.64 | 12.06 | 3.33 | 11.39 |
| deviation | | | | |
| Median | 3.22 | 97.12 | 4.12 | 102.70 |
| Minimum | 2.55 | 80.78 | 3.17 | 88.86 |
| Maximum | 4.49 | 118.10 | 12.06 | 118.77 |

| | | |
|---|---|---|
| p-value | <0.001* | <0.001* |

*Significant at the 5% level (Student's t test)

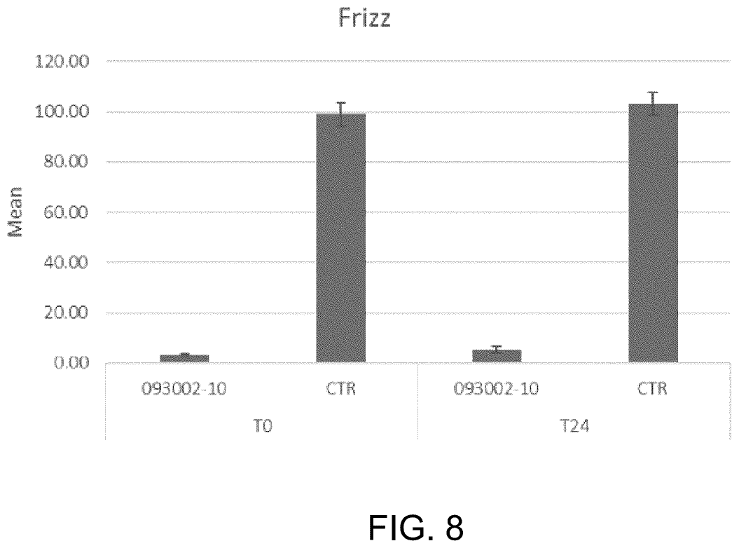

From the data presented in Table 4, FIG. 8 and the statistical analyses performed, it was possible to observed that the frizz for treatment 093002-10 was significantly lower compared to control (CTR) at time-points T0 and T24. No statistically significant difference was observed between time-points T0 and T24 for treatment 093002-10 and for CTR.

### Volume

Table 5 summarizes the statistical results obtained for each treatment for analysis of volume.

TABLE 5
Descriptive statistics and results of
the comparison between treatments.

| | |
|---|---|
| T0 | T24 |

| Statistics | 093002-10 | CTR | 093002-10 | CTR |
|---|---|---|---|---|
| | 22.36 | 146.81 | 26.07 | 145.45 |
| | 19.62 | 131.80 | 20.80 | 160.96 |
| | 17.43 | 134.07 | 25.09 | 161.65 |
| | 23.52 | 136.43 | 26.51 | 151.12 |
| | 21.54 | 137.33 | 23.51 | 151.71 |
| | 37.48 | 141.08 | 51.39 | 156.04 |
| Mean | 23.66 | 137.92 | 28.89 | 154.49 |
| Standard | 2.90 | 2.19 | 4.58 | 2.56 |
| error | | | | |
| 95% CI | [16.2; | [132.29; | [17.12; | [147.91; |
| | 31.11] | 143.55] | 40.66] | 161.06] |
| Standard | 7.10 | 5.36 | 11.22 | 6.27 |
| deviation | | | | |
| Median | 21.95 | 136.88 | 25.58 | 153.87 |
| Minimum | 17.43 | 131.80 | 20.80 | 145.45 |
| Maximum | 37.48 | 146.81 | 51.39 | 161.65 |
| p-value | <0.001* | | <0.001* | |

*Significant at the 5% level (Student's t test)

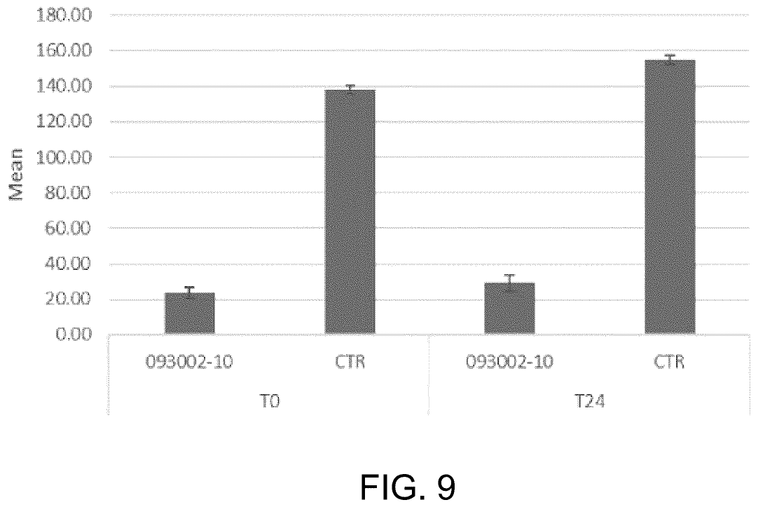

From the data presented in Table 5, FIG. 9 and the statistical analyses performed, it was possible to observed that the volume for treatment 093002-10 was significantly lower compared to control (CTR) at time-points T0 and T24. The volume at time-point T24 was significantly greater compared to T0 for treatment 093002-10 and for control (CTR).

### Split Ends

The table 6 summarizes the statistical results obtained for each treatment for analysis of Split ends.

TABLE 6
Descriptive statistics and results of
the comparison between treatments

| Treatment | 093002-10 | CTR |
|---|---|---|
| Statistics | 8 | 19 |
| | 10 | 20 |
| | 8 | 19 |
| | 11 | 14 |
| | 11 | 20 |
| | 8 | 25 |
| | 8 | 19 |
| | 8 | 19 |
| Sum | 72 | 155 |
| Mean | 9.0 | 19.4 |
| St. Error | 0.5 | 1.1 |
| 95% CI | [7.8; 10.2] | [16.9; 21.9] |
| St. Deviation | 1.4 | 3.0 |
| Median | 8.0 | 19.0 |
| Minimum | 8.0 | 14.0 |
| Maximum | 11.0 | 25.0 |
| p-value | <0.001* | — |

*Significant at 5% (Student's t test).

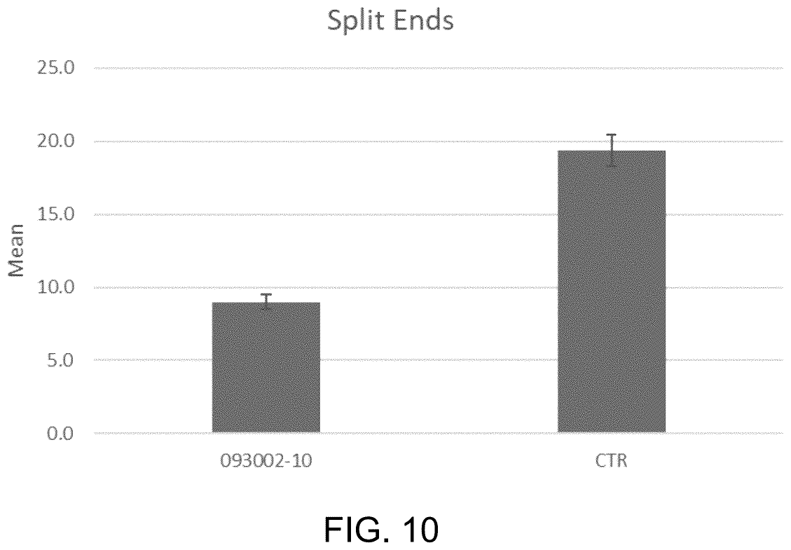

From the data presented in Table 6, FIG. 10 and the statistical analyses performed, it was possible to observed that a statistically significant reduction was observed in the number of split ends for treatment 092002-10 in comparison with CTR.

### Conclusion

Based on the results described above it is possible to conclude that the treatment complete air-dry cream composition presented definition and maintenance compared to the non-conditioning shampoo. The treatment complete air-dry cream composition has a similar motion compared to the non-conditioning shampoo. The treatment complete air-dry cream composition presented 54% split ends reduction and 2 times fewer split ends compared to the non-conditioning shampoo. The treatment complete air-dry cream composition presented 97% frizz reduction and 95% frizz control and 30 times less frizz compared to the non-conditioning shampoo. The treatment complete air-dry cream composition presented 83% volume reduction and 81% volume control and 6 times less volume compared to the non-conditioning shampoo. The treatment complete air-dry cream composition was shown to restore 8% of the natural luster of the treated hair.

### Example 2—Evaluation of a Complete Instant Recovery Serum Composition

### Summary

Hair Thermal analysis was done by DSC (differential scanning calorimetry). The higher the absolute value of enthalpy the higher the Keratin content of the hair obtained and compared between the following treatments: CTR: hair swatches washed with non-conditioning shampoo 20% SLES, and TRT1: hair swatches washed with a complete instant recovery serum composition (093002-09). Five measurements were done for each treatment.

Luster—Luster was evaluated by image analysis for the complete instant recovery serum composition. Each group containing three hair swatches was sorted as follows: Positive Control Group (CTRP): natural hair swatches washed with shampoo without conditioning agents SLES 20%; Negative control group (CTRN): natural hair swatches with the application of artificial sebum; and Group TRT: natural hair swatches washed with complete instant recovery serum composition (093002-09). The treatments above described were applied and the swatches were dried for 24 hours in an environment with humidity and temperature control. The dry swatches were photographed and evaluated by image analysis. The mean values of luster were statistically compared between the treatments and the summary of the results observed will be presented in the following section.

Split ends—Four Brazilian bleached hair swatches type II of 25 cm and 3 grams were prepared and separated into two different groups: CTR: swatches washed with a non-conditioning shampoo (20% SLES); and TRT1: natural hair swatches washed with complete instant recovery serum composition (093002-09). The swatches were treated for each group described above and brushed in 8 cycles of 200 brushing where the number of split ends were counted at the end of each cycle.

Combability—Five Brazilian bleached hair swatches type II of 25 cm and 3 grams were prepared and separated into two different groups: CTR: bleached hair swatches washed non-conditioning shampoo (20% SLES); and TRT1: bleached hair swatches washed with complete instant recovery serum composition (093002-09). The groups were evaluated in two steps: wet hair (just after washing the hair); and dry hair: after 24 hours drying overnight. The mean energy value was statistically compared between the treatments.

Color Retention Methodology—Four Brazilian natural hair swatches type II of 25 cm and 4 grams were prepared and separated into two different groups: CTR: hair swatches washed with non-conditioning shampoo 20% SLES; and TRT1: hair swatches washed with complete instant recovery serum composition (093002-09). The products were applied after dyeing the bleached hair swatches with a red dye (Garnier® 566) 20 times and evaluated at the following time-points: T0 Evaluation: bleached hair swatches; T5 Evaluation: after 5 consecutive washes; T10 Evaluation: after 10 consecutive washes; T15 Evaluation: after 15 consecutive washes; and T20 Evaluation: after 20 consecutive washes. At each time-point evaluated, the dry swatches were photographed, and the color evaluated in the CIELAB scale, with a Konica Minolta spectrophotometer (CM-2600d). The value of color difference ΔE2000 were obtained the following way: NW: color difference between the application of the dyeing product and bleached swatches; 5W color difference between the five washes and the bleached swatches; 10W color difference between the ten washes and the bleached swatches; 15W color difference between the fifteen washes and the bleached swatches; 20W color difference between the twenty washes and the bleached swatches. The ΔE2000 data were statistically compared, and the summary of results observed are presented in the following sections.

Results—After the statistical comparison it could be observed that the treatment complete instant recovery serum composition restores 100% of the natural hair luster, promoted hair Thermal protection compared with non-conditioning shampoo, promoted 40% split ends reduction and 2 times less split ends compared to the non-conditioning shampoo, presented 14% more combability compared to the non-conditioning shampoo, presented 19% more combability and compared to the non-conditioning shampoo, and maintained color for up to 20 washes.

### Introduction

Hair is a natural keratin fiber, a protein composed of a high degree of sulfur content present in the cysteine compounds. The mechanical properties observed in the hair depend, mainly, on its geometric structure and are very important in the evaluation of elasticity, softness and smoothness, combability, shine and so on. The evaluation of these properties by different methodologies can provide a better understanding of the real action of products on the hair to provide better marketing communications for these products. In the present study, 7 methodologies were done to evaluate treatment with a complete instant recovery serum composition compared to a non-conditioning shampoo: Thermal Analysis: to evaluate the enthalpy of keratin denaturation; Luster (shine): to evaluate shine on natural hair; Combability: to evaluate the easiness to comb the hair; Split ends: to evaluate split ends reduction; Color retention: to evaluate color fading due to multiple washing.

### Objective

The aim of the present study was to evaluate strength of the hair, thermal analysis Luster, Split ends, combability and color retention of hair compared to a non-conditioning shampoo.

### Methodology

The hair swatches were prepared and separated into two different groups: CTR: swatches washed with a non-conditioning shampoo (20% SLES); and TRT1: swatches washed with the complete instant recovery serum composition (093002-09). The groups were then treated with the non-conditioning shampoo before the start of the study to remove any residues hair might have as follow: 0.4 ml of non-conditioning shampoo per gram of hair was applied on the swatches and spread onto the wet hair. The swatch was massaged 6 times from the root to the ends (three times each side of the swatch); and then the swatch was rinsed for 30 seconds in tap water (5±1 L/min; 35±2° C.). After the step of removing the waste from a separate group as a control for later evaluation. The, the second group was washed with the Complete Instant Recovery Serum 1 as follows: swatches were rinsed for 30 seconds under a tap water (5±1 L/min; 35±2° C.) and the excess of water was removed; and with the wet swatches, 0.2 mL of Complete Instant Recovery Serum per gram of hair was applied on the swatches and spread. The swatch was massaged 6 times from the root to the tips (three times each side of the swatch).

Thermal Analysis to measure heat protection: After application, the locks were straightened 15 times at 230° C., then the hair was sent to a partner university where differential scanning calorimetry (DSC) analysis was performed.

Protection Against UV: After applying the treatments, the locks were left in natural drying (55±5% RH; 22±2° C.) and then prepared by the third institute for tryptophan evaluation.

Luster: After application left to dry in a humidity and temperature control chamber (55±5% RH; 22±2° C.) for complete drying. After a drying how locks were photographed.

Surface Analysis: After application left to dry in a humidity and temperature control chamber (55±5% RH; 22±2° C.) for complete drying.

Split ends: After application of the investigational products, the hair swatches were positioned in an automatic combing machine and brushed 1.600 times on their ends so as to promote the appearance of split ends.

Combability: For the moisture-loss group, the swatches were inserted in the moisture-loss apparatus, and the curves of Strength×Distance measured as represented in FIG. 5-b (CTR-wet and TRT-Wet. After the wet evaluation, the swatches were inserted in a room with controlled temperature and humidity (22±2° C.; 55±5% RH) and kept for 24 hours in this environment for complete drying of the swatches. After drying, the evaluation process was performed again.

Color retention: After application, the swatches were inserted in a room with controlled temperature and humidity (22±2° C.; 55±5% RH) and kept for 24 hours in this environment for complete drying of the swatches. At each interval of 5 washes, colorimetric measurements and photographic record were done and stored for later results presentation.

### Thermal Analysis to Measure Heat Protection

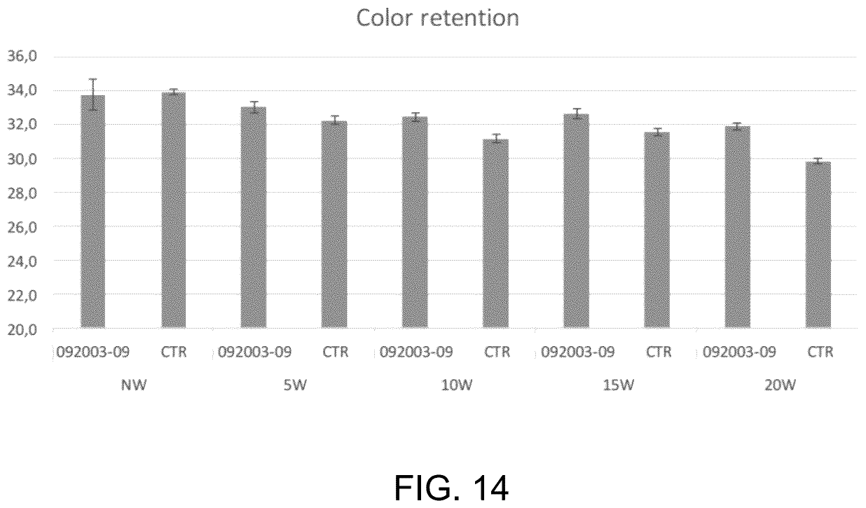

After products application, the swatches were dried using a hair drier and then flattened with flat iron 15 times at 450° F. The swatches were sent to an outsourced Institute for Differential scanning calorimetry (DSC) analysis as follows. For each group, 50 mg of hair were cut and inserted into five aluminum crucibles containing 10 mg each; and DSC (Mettler Toledo 823e) analyses were performed in an environment with temperature and humidity control (20-22° C. and 50-60%, respectively) with a heating rate of 10° C./min, temperature range of 40 to 300° C. and dynamic atmosphere of N2 (50 ml/min). FIG. 14 presents the heat transfer profiles of the two treatments evaluated and the enthalpy of the α-Keratin denaturation peak. The higher the absolute value of enthalpy the higher the Keratin content of the hair. This means that the process of flat ironing the hair at 450° F. will be less damaged for the treatment that presents a higher enthalpy value (absolute value) after the DSC analysis. For the five enthalpy measurements the mean value was statistically compared by Student t-test of unpaired samples with a 95% confidence interval. The results can be seen in the next chapter and the raw data and statistical output in the appendix of this report. The data of enthalpy of vaporization were statistically compared and are presented below.

### Luster

Images were photographed in a luster chamber. The images obtained are treated and analyzed with the software program ImageJ® in order to obtain the diffuse and specular light profiles of each image (See FIG. 4) to calculate luster as presented in equation below. From the diffuse reflection and specular reflection curves of the total intensity profile presented in FIG. 6, the areas under the curve of both reflections are calculated from numeric methods of approximation and used for the calculation

$${B}_{ALL} = ( \frac{{D}_{Stamm}}{{S}_{Gaussian}} ) \times \frac{{I}_{MAX}}{{W}_{1 / 2}}$$

of luster in which B_{ALL} is the value of luster defined by the adjusted equation above, where D_{Stamm} is the diffuse light reflection, S_{Gaussian} is the specular light reflection, IMAX is the maximum light intensity and w1/2 is the maximum width at half-height, or the width of specular reflection shown in FIG. 5.

### Split Ends

After application of the investigational products, the hair swatches were positioned in an automatic combing machine and brushed 16000 times on their ends so as to promote the appearance of split ends, and at each cycle of 200 brushings, the split ends were counted with the aid of a magnifying glass. The value obtained was tabulated and sent for statistical analysis as described below.

### Combability

After treatment application, the swatches were put in a comb support holder attached to an INSTRON 5565 device and combed while the device recorded the resistance to combing. The energy is calculated through the integral of the curves of strength vs. distance recorded for each swatch and the mean value for five measurements of energy were analyzed as represented below.

### Color Retention

Color measurements were be performed through the equipment Spectrophotometer Konica Minolta Sensing INC CM-2600D. Colors can be classified by their hue, luminosity and saturation. Any color can therefore be characterized in a space where geometric coordinates can help to determine brightness, dominant coloring and saturation level. Several spaces of different colors exist, mathematic links making possible the conversion of the coordinates from one to another. Chromameter shows three color range values (MINOLTA, 2007): L*—Luminosity (dark-light) and their values range from 0 to 100. The bigger the L* values, the lighter the sample will be a*—the chromatic green/red axis, the values range from −60 to +60. The more positive the a* values, the redder the sample will be; and b*—the chromatic axis blue-yellow and their values range from −60 to +60. The more positive the b* values, the more yellow the sample will be. To analyze color-Retention attribute after multiple washes, the differences of ΔE2000 (T″X″1—T0; X means non-washing or 5, 10, 15 and 20 washes) were used for the time-points analyzed. The lower the value of ΔE2000 the closer the color to the bleached hair. Therefore, fading will be represented by the lowering of the color difference. However, to observe no difference among the dyed hair and the bleached hair the color difference must be under 3.0, i.e., ΔE2000=3.0. To evaluate if the product maintained the hair color after multiple washes, the following time-points were evaluated: T0 Evaluation: Swatches bleached and evaluated before application of dye color 566; T1 Evaluation: Swatches evaluated after application of dye color 566 and before wash; T5 Evaluation: Swatches evaluated after 5 consecutive washes; T10 Evaluation: Swatches evaluated after 10 consecutive washes; T15 Evaluation: Swatches evaluated after 15 consecutive washes; and T20 Evaluation: Swatches evaluated after 20 consecutive washes. For each colorimetrically evaluated time-point (Treatment and 20% SLES), a photographic record was done for the visual presentation of the results.

### Analysis of Results

### Statistical Analysis

Exploratory data analysis was performed (mean, standard error, standard deviation, minimum, median, maximum, 95% Confidence Interval and charts). Treatments were compared through Student's t test. Software used was XLSTAT 2019 and MINITAB 14. Confidence level: 95%.

### Analysis of Results

The equations shown below can be used, although a difference is always observed, to estimate the percentage of breakage reduction and the how much less breakage compared to 20% SLES:

$$\% BR = ( 1 - \frac{TRT}{SLES} ) \times 100 \\ Number of times = {( \frac{TRT}{SLES} )}^{- 1}$$

### Results

### Thermal Analysis to Measure Heat Protection

Table 7 presents the results observed and descriptive statistics by treatment for analysis of protection Thermal.

TABLE 7
Mean and Standard Error by treatment evaluated

| Statistics | 093002-09 | CTR |
|---|---|---|
| | 63.70 | 57.60 |
| | 86.20 | 65.80 |
| | 78.70 | 69.00 |
| | 88.70 | 72.80 |
| | 84.20 | 73.10 |
| Mean | 80.3 | 67.7 |
| St. Error | 4.5 | 2.9 |
| 95% CI | [67.9; 92.7] | [59.7; 75.6] |
| St. Deviation | 10.0 | 6.4 |
| Median | 84.2 | 69.0 |
| Minimum | 63.7 | 57.6 |
| Maximum | 88.7 | 73.1 |

| | |
|---|---|
| p-value | 0.049* |

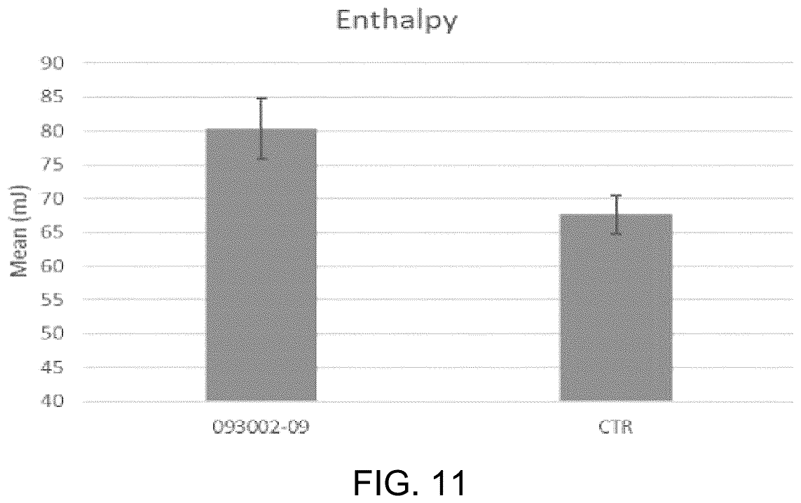

From the data presented in Table 7, FIG. 11 and the statistical analyses performed, it was possible to observe that the enthalpy was higher for treatment 093002-09 in comparison with CTR. Luster

Table 8 presents the results observed and descriptive statistics by treatment for analysis of luster.

TABLE 8
Mean and Standard Error by treatment evaluated

| Statistics | CTRP | CTRN | 093002-09 |
|---|---|---|---|
| Mean | 5.89 | 2.05 | 6.00 |
| St. Error | 0.01 | 0.00 | 0.01 |
| 95% CI | [5.87; 5.9] | [2.04; 2.07] | [5.98; 6.01] |
| St. Dev. | 0.02 | 0.02 | 0.03 |
| Median | 5.88 | 2.06 | 6.00 |
| Minimum | 5.84 | 2.02 | 5.95 |
| Maximum | 5.95 | 2.07 | 6.04 |

| Comparison | P-value | Conclusion |
|---|---|---|
| 093002-09 vs CTRN | <0.001* | Better luster for 093002-09 |
| 093002-09 vs CTRP | <0.001* | Better luster for 093002-09 |
| CTRP vs CTRN | <0.001* | Better luster for CTRP |

*Significant at 5% (Fisher's LSD test).

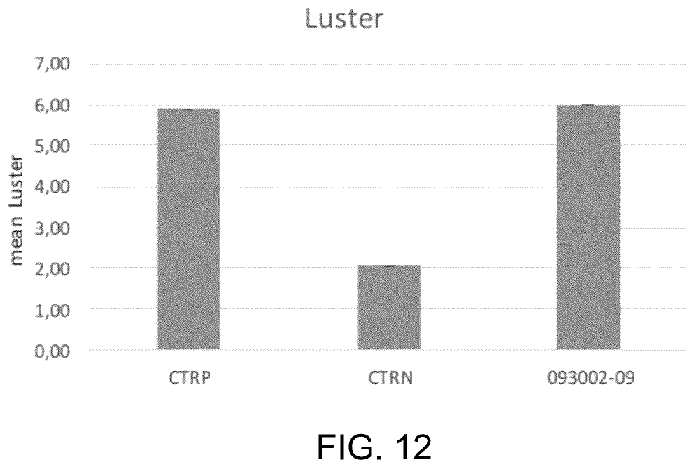

From the data presented in Table 8, FIG. 12 and the statistical analyses performed, it was possible to observed that: The treatment 093002-09 presented a statistically greater luster compared to CTRP and CTRN. The treatment CTRP presented a statistically greater luster compared to CTRN. It possible to conclude that the treatment 093002-09 restores 100% of the natural luster of the hair and 2.9 times more luster compared to the sebum treatment.

### Split Ends

Table 9 presents the results observed and descriptive statistics by treatment for analysis of Split Ends.

TABLE 9
Mean and Standard Error by treatment evaluated

| Statistics | 093002-09 | CTR |
|---|---|---|
| | 14 | 19 |
| | 12 | 20 |
| | 9 | 19 |
| | 14 | 14 |
| | 11 | 20 |
| | 12 | 25 |
| | 10 | 19 |
| | 11 | 19 |
| Sum | 93 | 155 |
| Mean | 11.6 | 19.4 |
| St. Error | 0.6 | 1.1 |
| 95% CI | [10.1; 13.1] | [16.9; 21.9] |
| St. Deviation | 1.8 | 3.0 |
| Median | 11.5 | 19.0 |
| Minimum | 9.0 | 14.0 |
| Maximum | 14.0 | 25.0 |
| p-value | <0.001* | <0.001* |

*Significant at 5% (Student's t test)

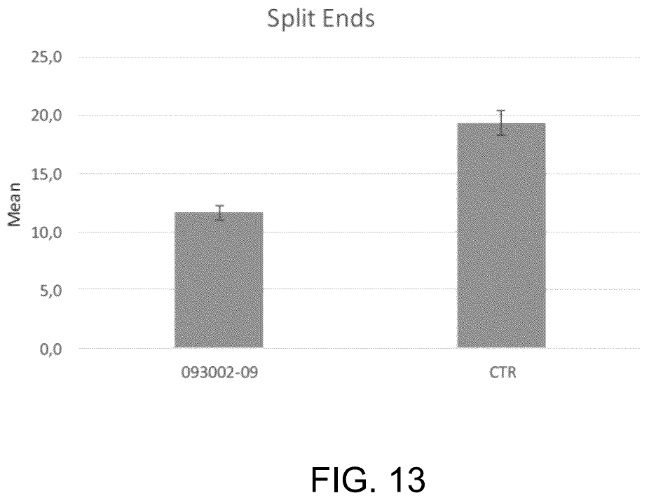

From the data presented in Table 9, FIG. 13 and the statistical analyses performed, it was possible to observed that a statistically significant reduction was observed in number of split ends for treatments, 093002-09 in comparison with CTR.

### Color Retention

Table 10 presents the results observed and descriptive statistics by treatment for analysis of color retention.

TABLE 10
Mean and Standard Error by treatment evaluated

| | NW | | 5 W | | 10 W | | 15 W | | 20 W | |
|---|---|---|---|---|---|---|---|---|---|---|
| Statistics | 092003-09 | ^{CTR} | 092003-09 | ^{CTR} | 092003-09 | ^{CTR} | 092003-09 | ^{CTR} | 092003-09 | CTR |
| Mean | 33.74 | 33.91 | 33.03 | 32.25 | 32.45 | 31.17 | 32.62 | 31.55 | 31.87 | 29.83 |
| St. Error | 0.90 | 0.20 | 0.32 | 0.24 | 0.25 | 0.22 | 0.28 | 0.23 | 0.18 | 0.19 |
| 95% CI | [31.92 ± | [33.52 ± | [32.37 ± | [31.77 ± | [31.95 ± | [30.72 ± | [32.06 ± | [31.09 ± | [31.51 ± | [29.44 ± |
| | 35.56] | 34.3] | 33.69] | 32.73] | 32.95] | 31.62] | 33.18] | 32.01] | 32.23] | 30.22] |
| St. Dev. | 5.68 | 1.23 | 2.05 | 1.51 | 1.56 | 1.40 | 1.75 | 1.45 | 1.13 | 1.23 |
| Median | 34.37 | 33.80 | 33.43 | 32.27 | 32.92 | 31.24 | 32.80 | 31.57 | 32.03 | 29.94 |
| Minimum | 0.00 | 31.37 | 29.58 | 28.80 | 29.84 | 28.40 | 29.56 | 28.42 | 29.87 | 27.26 |
| Maximum | 37.05 | 36.19 | 36.31 | 34.82 | 34.75 | 33.75 | 35.11 | 34.03 | 33.32 | 32.13 |

| | | | | | |
|---|---|---|---|---|---|
| p-value | 0.848 | 0.056 | <0.001* | 0.004* | <0.001* |

From the data presented in Table 10, FIG. 14 and the statistical analyses performed, it was possible to observed that the mean was significantly lower for 10W, 15W and 20W in comparison with NW for treatment 092003-09. The mean was significantly lower for 20W in comparison with 5W for treatment 092003-09. No statistically significant difference was observed between any other washes.

### Conclusion

According to the methodologies used to evaluate the treatment complete instant recovery serum composition, it is possible to conclude that treatment restores 100% of the natural luster and offers 3% more luster to the hair. The treatment promoted hair Thermal protection in compared with non-conditioning shampoo. The treatment promoted 40% split ends reduction and 2 times fewer split ends compared to the non-conditioning shampoo. No statistically significant difference in energy/cm was observed for the treatment in comparison with non-conditioning shampoo. On the dry hair, the treatment presented 19% more combability and was easier to comb compared to the non-conditioning shampoo. The treatment maintains color up to 20 washes.

### Example 3—UV Protection Evaluation of a Complete Instant Recovery Serum Composition and Complete Air-Dry Cream Composition

### Summary

Two Brazilian natural hair swatches type II of 20 cm and 3 grams were prepared and separated into four different groups: CTRL: without exposition to UV radiation; 2. CTRL negative: 96 hour of UV radiation exposure; TRAT1: complete instant recovery serum composition (093002-09) application followed by 96 hours of UV radiation exposure; and TRAT2: complete air-dry cream composition (093002-10) application followed by 96 hours of UV radiation exposure. After the UV radiation exposure, the hair swatches were analyzed by a spectrophotometer Fluorolog-Jobin Yvon Horibe FL3-12 with a monochromator Xenon lamp to evaluate the tryptophan (Trp) content of the hair. Three measurements were done by hair swatch and the mean value compared statistically among the groups. The closer the tryptophan content of the treatment compared to CTRL the higher the UV protection. For the two treatments tested by this technique it was possible to observe the following results: the complete instant recovery serum composition presented an UV protection to the hair of, approximately, 96%. The complete air-dry cream composition presented an UV protection to the hair of about 71%.

### Introduction

It is well established in the literature that ultraviolet (UV) and visible lights can cause damage to hair fibers. The photochemical degradation of the fibers occurs both in the keratin and in the melanin responsible for the hair color. Melanin also provides a certain degree of photoprotection to the fibers absorbing and dissipating radiation from short wavelengths (λ<320 nm). One of the main components of melanin is the tryptophan (Trp) amino acid. When exposed to electromagnetic waves of high energy (short wavelengths) it degrades in several steps, forming the radical Trp, which converts to N-formyl kynurenine and finally produces kynurenine. Thus, the relative concentration of Trp in the Caucasian hair fiber may be an intrinsic reference to measure the degree of photodegradation. Therefore, one can use this photochemistry property to evaluate the Trp degradation process as a function of time when exposure to solar radiation.

### Objective

The objective of the present study was to evaluate the UV protection provided after the application of a complete air-dry cream composition and a complete instant recovery serum composition.

### Study Protocol

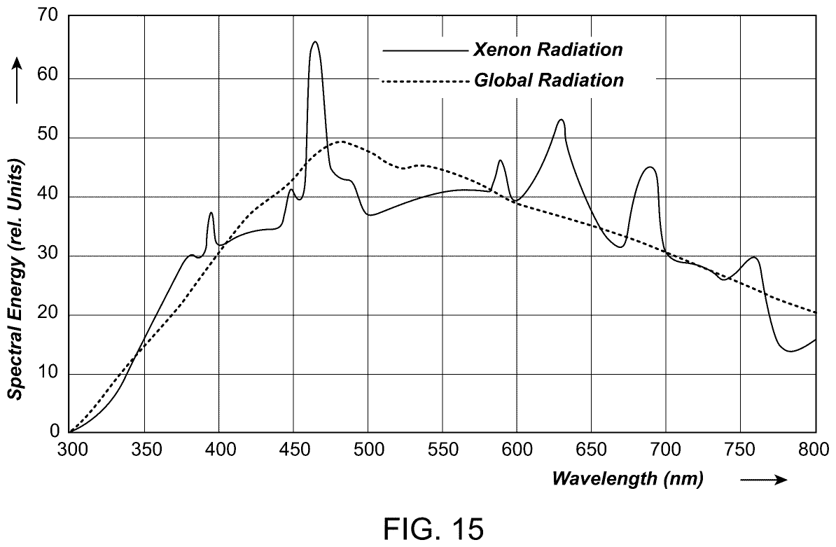

Two Brazilian natural hair swatches type II of 20 cm and 3 grams were prepared and separated into four different groups: CTRL: without exposition to UV radiation; 2. CTRL negative: 96 hour of UV radiation exposure; TRAT1: complete instant recovery serum composition (093002-09) application followed by 96 hours of UV radiation exposure; and TRAT2: complete air-dry cream composition (093002-10) application followed by 96 hours of UV radiation exposure. Prior to the test, the groups were then treated with the non-conditioning shampoo to remove any residues the hair might have: 0.4 ml of non-conditioning shampoo per gram of hair was applied on the swatches and spread onto the wet hair. The swatch was massaged 6 times from the root to the ends (three times each side of the swatch); Then, the swatch was rinsed for 30 seconds in tap water (5±1 L/min; 35±2° C.); After that, the treatments were applied as follow: The swatches were rinsed for 30 seconds under a tap water (5±1 L/min; 35±2° C.) and the excess of water was removed; With the wet swatches, 0.2 ml of treatment per gram of hair was applied on the swatches and spread. The swatch was massaged 6 times from the root to the tips (three times each side of the swatch). The swatches were left to dry overnight (55±5% RH; 22±2° C.) and then exposed to UV radiation for 96 hours. Table 11 and FIG. 15 show, the equipment configuration and the spectral profile of the lamp respectively.

TABLE 11
Atlas Weather-Ometer specifications

| | |
|---|---|
| Radiation source | 6500 W Xenon lamp |
| Aging cycle | Without insulation and rain exposure |
| Control | Irradiance of 0.35 W/m² at 340 nm |
| Total density energy | 2641 mW/cm² |

After the irradiation of the hair swatches, they tryptophan content were analyzed with a spectrophotometer Fluorolog-Jobin Yvon Horibe FL3-12. FIG. 216 shows the spectral measurements for each evaluated group.

### Analysis of Results

### Statistical Analysis

Exploratory data analysis was performed (mean, standard error, standard deviation, minimum, median, maximum, 95% Confidence Interval and charts). Treatments were compared through Student's t test. Software used was XLSTAT 2019 and MINITAB 14. Confidence level: 95%.

### Results

The equation below was used to calculate the UV protection of the treatments whenever a difference was observed when compared to the negative (CTRLN) and positive (CTRLP) control:

$$[ 1 - ( \frac{( CTRLP - CTRLN ) - ( TRAT - CTRLN )}{( CTRLP - CTRLN )} ) ] * 100$$

Table 12 shows the results of 6 measurements of tryptophan emission at 325 nm (maximum emission).

TABLE 12
Tryptophan emission at 325 nm by treatment

| Measurement | CTRL | CTRL negative | TRAT1 | TRAT2 |
|---|---|---|---|---|
| 1 | 28.729 | 20.361 | 29.955 | 26.637 |
| 2 | 29.299 | 20.406 | 30.423 | 27.984 |
| 3 | 29.628 | 20.602 | 29.553 | 27.857 |
| 4 | 28.201 | 21.680 | 25.343 | 24.871 |
| 5 | 27.500 | 19.162 | 26.674 | 24.623 |
| 6 | 27.644 | 19.866 | 27.089 | 25.048 |
| Mean tryptophan value | 28.501 | 20.346 | 28.173 | 26.170 |
| STD deviation | 0.870 | 0.834 | 2.077 | 1.558 |

### Conclusion

According to the results presented in table 12 and the statistical analysis performed it was possible to observe that treatments TRAT1 and TRAT2 did not present a significant statistical difference compared to CTRL (hair without exposition to UV radiation). Treatments TRAT1, TRAT2 and CTRL presented a higher tryptophan content compared to CTRL negative (unprotected hair exposure to UV radiation). Using the equation above, it was possible to observe the following percentage of protection: the complete instant recovery serum composition (TRAT1) presented an UV protection to the hair of, approximately, 96%; the complete air-dry cream composition (TRAT2) presented an UV protection to the hair of, approximately, 71%.

### Example 4—Evaluation of Complete Pre-Wash Scalp Oil

### Summary

Reduction in hair fall associated with breakage—Four Brazilian natural hair swatches type III of 25 cm and 3 grams were prepared and separated into two different groups: CTR: swatches washed with a non-conditioning shampoo (20% SLES); and TRT1: natural hair swatches washed with complete pre-wash scalp oil composition (093002-08). The swatches were treated for each group described above and brushed in 8 cycles of 1000 brushing where the number of broken fibers were counted at the end of each cycle.
Surface Analysis to measure damage restoration—The present study evaluated the restoration using scanning electron microscopy for two treatments. Two groups containing bleached hair swatches were separated by treatment as described below: CTR: swatches washed with a non-conditioning shampoo (20% SLES); TRT1: natural hair swatches washed with complete pre-wash scalp oil composition (093002-08)
Split ends—Four Brazilian bleached hair swatches type II of 25 cm and 3 grams were prepared and separated into two different groups: CTR: swatches washed with a non-conditioning shampoo (20% SLES); and TRT1: natural hair swatches washed with complete pre-wash scalp oil composition (093002-08). The swatches were treated for each group described above and brushed in 8 cycles of 200 brushing where the number of split ends were counted at the end of each cycle.
Tensile test—To evaluate hair strength, 50 hair fibers were collected from each treatment and analyzed by INSTRON (5565), where the stress-strain behavior was recorded for each hair fiber and the maximum stress and Young modulus obtained and stored for further comparison.
Restoration Methodology—To evaluate hair restoration, four methodologies were used: luster; frizz and volume; combability; and hydrophobicity. Two groups of hair swatches were separated by treatment evaluated as described below: Control Group (CTR): bleached hair swatches washed with non-conditioning shampoo 20% SLES. Group TRT: bleached hair swatches washed with the Intensive Complete pre-wash scalp oil composition

### Results

After the statistical comparison it could be observed that: Treatment with the complete pre-wash scalp oil composition presented 93% Breakage reduction and 15 times less breakage compared to the non-conditioning shampoo. Treatment with the complete pre-wash scalp oil composition presented hair surface protection compared to the non-conditioning shampoo due to surface oil deposition. Therefore, it was possible to observe 94% damage repair and 19 times less damage compared to the non-conditioning shampoo. Treatment with the complete pre-wash scalp oil composition presented 57% split ends reduction and 2 times fewer split ends compared to the non-conditioning shampoo. Treatment with the complete pre-wash scalp oil composition presented 12% more strength compared to the non-conditioning shampoo. The complete pre-wash scalp oil composition product promoted 100% luster (shine) restoration when compared to the positive and negative control groups; 98% frizz reduction and 97% frizz control and 41 times less frizz compared to the non-conditioning shampoo; 82% volume reduction and 84% volume control and 6 times less volume compared to the non-conditioning shampoo; 30% more combability on the wet hair compared to the non-conditioning shampoo; 72% more combability and was 4 times easier to comb on the dry hair compared to the non-conditioning shampoo; and 8% hydrophobicity restore when compared to the positive and negative control.

### Introduction

Hair is a natural keratin fiber, a protein composed of a high degree of sulfur content present in the cysteine compounds. The mechanical properties observed in the hair depend, mainly, on its geometric structure and are very important in the evaluation of elasticity, softness and smoothness, combability, shine and so on. The evaluation of these properties by different methodologies can provide a better understanding of the real action of products on the hair to provide better marketing communications for these products. In the present study, 4 methodologies were done to evaluate the treatment Shampoo plus Conditioner Embody Volumizing compared to a non-conditioning shampoo hair breakage resistance: to evaluate the reduction in hair breakage due to multiple brushing; surface analysis to evaluate damage repair via SEM; split ends to evaluate split ends reduction; tensile strength to evaluate hair strength; and restoration to evaluate hair damage restoration.

### Objective

The objective of the current study was to evaluate the hair breakage resistance, damage restoration. surface analysis, reduction in split ends and strength for the treatment complete pre-wash oil composition in comparison with a non-conditioning shampoo.

### Methodology

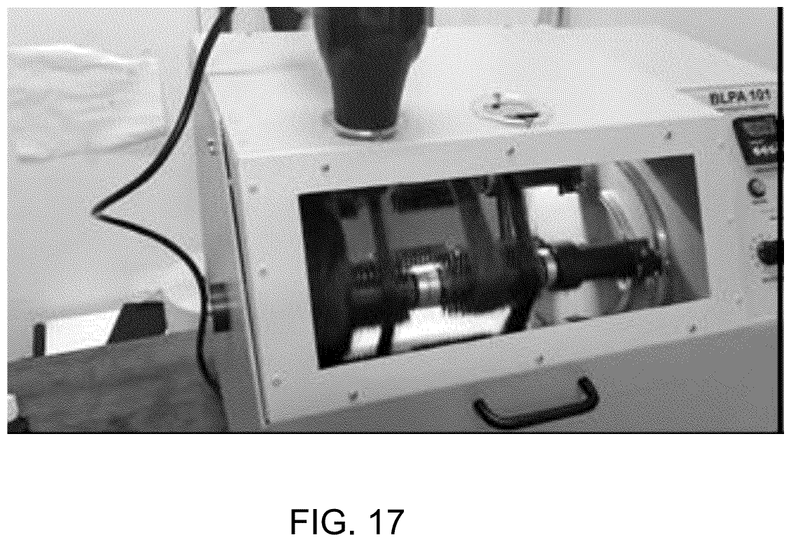

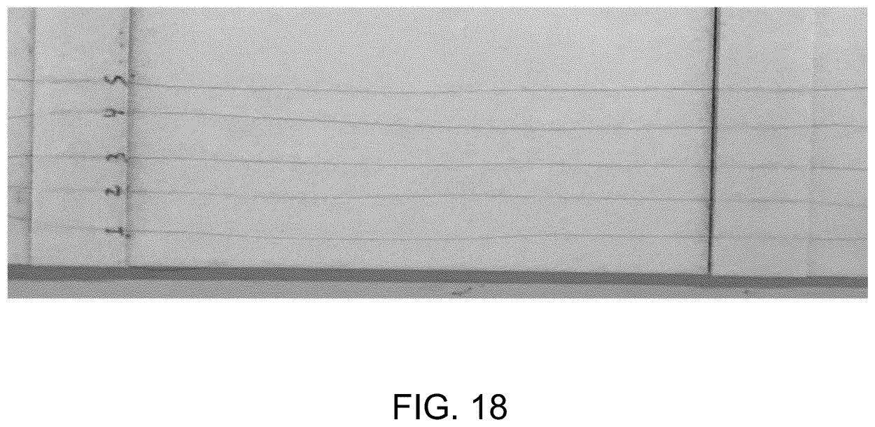

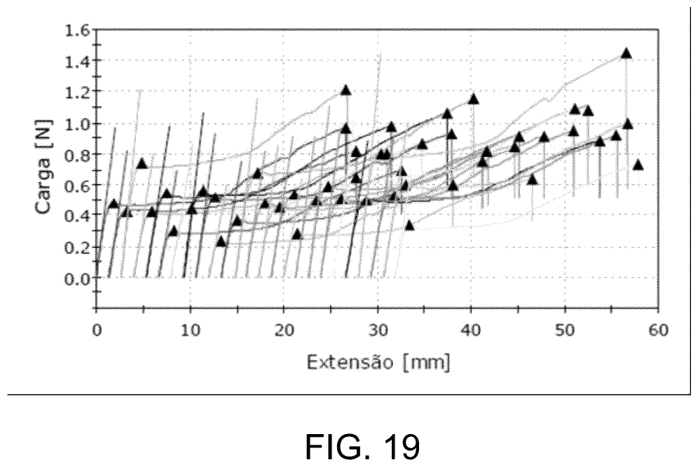

Hair swatches were prepared and separated into two different groups as follows: CTR: swatches washed with a non-conditioning shampoo (20% SLES); 2. TRT1: swatches washed with the complete pre-wash scalp oil composition (093002-08). The groups were then treated with the non-conditioning shampoo before the start of the study to remove any residues hair might have as follows: 0.4 ml of non-conditioning shampoo per gram of hair was applied on the swatches and spread onto the wet hair. The swatch was massaged 6 times from the root to the ends (three times each side of the swatch); Then, the swatch was rinsed for 30 seconds in tap water (5±1 L/min; 35±2° C.). After the step of removing the waste from a separate group as a control for later evaluation. The second group was washed with the complete pre-wash scalp oil composition as follows: The swatches were rinsed for 30 seconds under a tap water (5±1 L/min; 35±2° C.) and the excess of water was removed; With the wet swatches, 0.2 mL of complete pre-wash scalp oil composition per gram of hair was applied on the swatches and spread. The swatch was massaged 6 times from the root to the tips (three times each side of the swatch).
Breakage: After the step of removal of the residues the first group was inserted into the breakage machine and the test started. The second group left to dry in a humidity and temperature control chamber (55±5% RH; 22±2° C.) until the test with the first group was complete.
Surface Analysis: After application left to dry in a humidity and temperature control chamber (55±5% RH; 22±2° C.) for complete drying.
Tensile test: After application, 50 hair fibers were collected for mechanical analysis.
Split ends: After application of the investigational products, the hair swatches were positioned in an automatic combing machine and brushed 1.600 times on their ends so as to promote the appearance of split ends.
Restoration: After application left to dry in a humidity and temperature control chamber (55±5% RH; 22±2° C.) for complete drying and evaluated on shine, frizz, combability and hydrophobicity.
Evaluation of Breakage Resistance: After the application of the treatments in their respective groups, the swatches were positioned in the automatic combing machine (FIG. 17) with a professional hair dryer attached to it and brushed 10000 times in cycles of 1000 brushings. At the end of each cycle, the broken hair fibers were manually counted, and the amount stored for further comparison.
Evaluation of Surface Analysis to measure damage restoration—The images were performed with the Scanning Electron Microscope with electrons gun by field emission, FEG-SEM, Zeiss model Supra 35. The acquisition of these images has as objective to analyze the surface profile of the hair fibers after the treatments previously described: how the products protect the hair fibers from damages. For that, six strands of each swatch treated with SH1 and SH2 were randomly taken and put in aluminum “stubs” with graphite tape, so that the electrons can flow through the fibers without damaging them. The strands are then scanned by the microscope and six images (one for each strand) with amplitude of 2000× of magnification are done, highlighting the profile presented by the scanning. In addition, an illustrative image of 10000× of magnification is performed. The images are evaluated with the ImageJ™ in which the damages are manually selected by a trained technician and a macro, internally developed, reads and quantifies the regions of damage selected. In the end, the damage area selected is divided by the total area analyzed and the percentage of damage for the image analyzed is quantified. The data for six swatches were analyzed statistically and will be presented in the next section.
Split ends—After application of the investigational products, the hair swatches were positioned in an automatic combing machine (FIG. 17) and brushed 1.6000 times on their ends so as to promote the appearance of split ends, and at each cycle of 200 brushings, the split ends were counted with the aid of a magnifying glass. The value obtained was tabulated and sent for statistical analysis as described below.
Tensile Strength—After application of all treatments identified above, the hair fibers were prepared and the test was conducted the following way: So that the fibers did not come lose during the Tensile test, they were glued with adhesive tape and Super Bonder® glue in the extremities as presented in FIG. 18. Then, the diameters were measured with a micrometer Mitutoyo in three difference regions of the fibers. After measurement of the diameter, the fibers are put in the Instron (5565) device, and the mean diameter of three measurements performed are inserted in the software. Then, the test is conducted, and the parameters are automatically calculated for each fiber. The curves Load vs. Extension may be seen in FIG. 19. For each group, 50 measurements of each one of the parameters are performed and the mean value of each group was statistically compared.
Total Restoration—To evaluate hair restoration, four methodologies were used:
Luster—The images were photographed in a luster chamber developed internally after each procedure of the treatment. The images obtained were treated and analyzed with the software program ImageJ® in order to obtain the diffuse and specular light profiles of each image (FIG. 4) to calculate luster as presented in the equation below. From the diffuse reflection and specular reflection curves of the total intensity profile presented in FIG. 6, the areas under the curve of both reflections are calculated from numeric methods of approximation and used for the calculation of luster in which BALL is the value of luster defined by the adjusted equation, DStamm is the diffuse light reflection, SGaussian is the specular light reflection, IMAX is the maximum light intensity and w1/2 is the maximum width at half-height, or the width of specular reflection shown in FIG. 4.
Frizz—The quantification of frizz and volume of the images obtained was done with the software ImageJ® by using an internally developed macro, in which the frizz can be separated from the body of the hair sample and the area used to assess the 2D volume of the hair swatch (See FIG. 3). As mentioned, the frizz phenomena appear during the natural drying process of the hair swatches and it becomes more emphasized in areas where the relative humidity are more elevated. Through the appropriate methodology, it is possible to identify both reduction and control of frizz and volume provided by a certain treatment. The images obtained during the study were stored and the results of frizz and volume were sent for statistical comparison as described in the following section.
Combability—After treatment application, the swatches were put in a comb support holder attached to an INSTRON 5565 device and combed while the device recorded the resistance to combing. The energy is calculated through the integral of the curves of strength vs. distance recorded for each swatch and the mean value for five measurements of energy were analyzed as represented below.
Hydrophobicity—After washing the swatches with respective test products and 20% SLES, the swatches were placed to dry for 24 hours in a room with controlled temperature and humidity (22±2° C.; (50±5% RH). After drying, the hair swatches were horizontally placed so the fibers are aligned, and the water drops placed in different areas of the hair swatch. The test was filmed for all treatments and the angle of the drop against the hair surface and remaining time over the hair was computed to check the formation of a protection film over the hair.

### Statistical Analysis

Exploratory data analysis was performed (mean, standard error, standard deviation, minimum, median, maximum, 95% Confidence Interval and charts). Treatments were compared through ANOVA followed by LSD multiple comparison test. Software: XLSTAT 2021 and MINITAB 14. Confidence level: 95%.

### Results

Breakage—Table 13 presents the results observed and descriptive statistics by treatment for analysis of number of broken hair fibers.

TABLE 13
Mean and Standard Error by treatment evaluated

| Statistics | 093002-08 | CTR |
|---|---|---|
| | 3 | 46 |
| | 1 | 40 |
| | 3 | 54 |
| | 2 | 30 |
| | 1 | 33 |
| | 4 | 41 |
| | 3 | 59 |
| | 7 | 47 |
| Sum | 24.0 | 350 |
| Mean | 3.0 | 43.8 |
| St. Error | 0.7 | 3.5 |
| 95% CI | [1.4; 4.6] | [35.5; 52] |
| St. Deviation | 1.9 | 9.9 |
| Median | 3.0 | 43.5 |
| Minimum | 1.0 | 30.0 |
| Maximum | 7.0 | 59.0 |

| | |
|---|---|
| p-value | <0.001* |

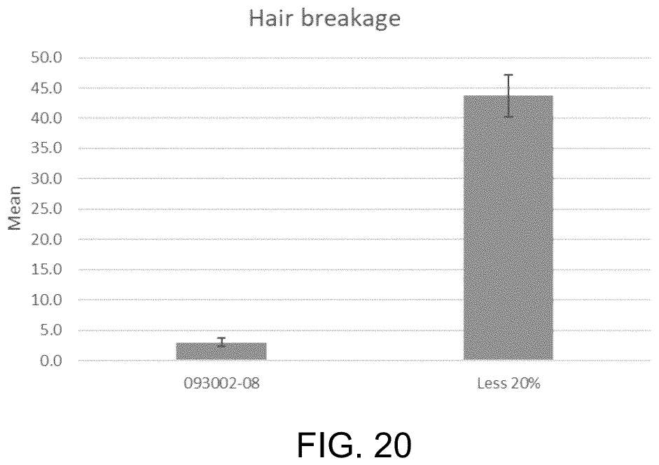

From the data presented in Table 13, FIG. 20 and the statistical analyses performed, it was possible to observed that the number of broken hair strands was lower for treatment 093002-08 in comparison with control CTR.

Surface Analysis—Table 14 presents the results observed and descriptive statistics by treatment for analysis of restoration.

TABLE 14
Mean and Standard Error by treatment evaluated

| Statistics | 093002-08 | CTR |
|---|---|---|
| | 0.00 | 8.45 |
| | 0.97 | 12.42 |
| | 0.95 | 10.11 |
| | 1.57 | 11.85 |
| | 0.00 | 12.34 |
| | 0.00 | 10.20 |
| Mean | 0.6 | 10.9 |
| St. Error | 0.3 | 0.6 |
| 95% CI | [−0.1; 1.3] | [9.2; 12.5] |
| St. Deviation | 0.7 | 1.6 |
| Median | 0.5 | 11.0 |
| Minimum | 0.0 | 8.5 |
| Maximum | 1.6 | 12.4 |

| | |
|---|---|
| p-value | <0.001* |

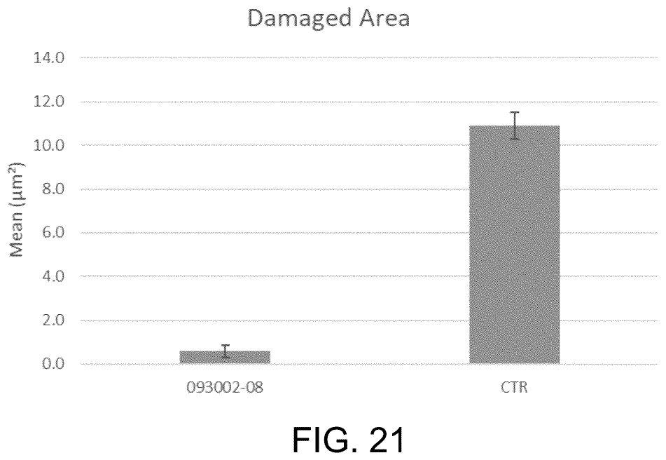

From the statistical comparison presented in Table 14 and FIG. 21, it was possible to verify that a statistically significant lower damaged area was observed for treatment 093002-08 in comparison with CTR.
Split ends—Table 15 presents the results observed and descriptive statistics by treatment for analysis of split ends.

TABLE 15
Mean and Standard Error by treatment evaluated

| Statistics | 093002-08 | CTR |
|---|---|---|
| | 10 | 19 |
| | 8 | 20 |
| | 11 | 19 |
| | 6 | 14 |
| | 8 | 20 |
| | 7 | 25 |
| | 9 | 19 |
| | 7 | 19 |
| Sum | 66 | 155 |
| Mean | 8.3 | 19.4 |
| St. Error | 0.6 | 1.1 |
| 95% CI | [6.9; 9.6] | [16.9; 21.9] |
| St. Deviation | 1.7 | 3.0 |
| Median | 8.0 | 19.0 |
| Minimum | 6.0 | 14.0 |
| Maximum | 11.0 | 25.0 |
| p-value | <0.001* | — |

*Significant at 5% (Student's t test)

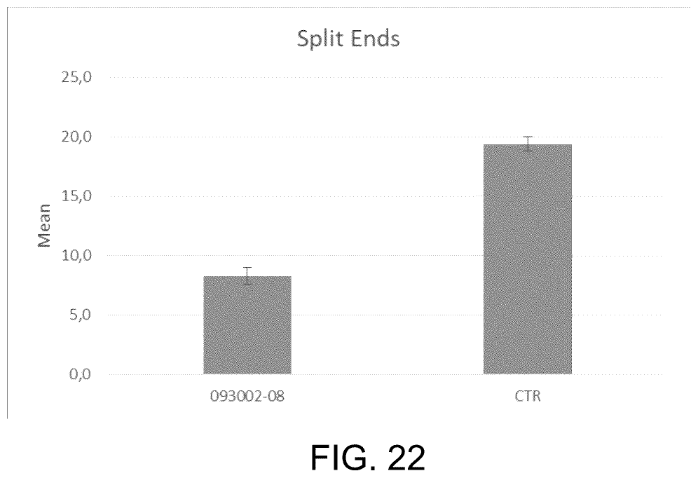

From the statistical comparison presented in Table 15 and FIG. 22 it was possible to verify that a statistically significant reduction was observed in number of split ends for treatments 093002-03/04, 093002-09 and 093002-08 in comparison with CTR.

Restoration—Luster—Table 16 presents the results observed and descriptive statistics by treatment for analysis of luster

TABLE 16
Mean and Standard Error by treatment evaluated

| Statistics | CTRP | CTRN | 093002-08 |
|---|---|---|---|
| Mean | 5.19 | 4.28 | 6.09 |
| St. Error | 0.29 | 0.17 | 0.10 |
| 95% CI | [4.57; 5.8] | [3.91; 4.66] | [5.86; 6.31] |
| St. Dev. | 1.11 | 0.68 | 0.40 |
| Median | 4.95 | 4.14 | 6.08 |
| Minimum | 3.59 | 3.18 | 5.41 |
| Maximum | 7.61 | 5.81 | 6.58 |

| Comparison | P-value | Conclusion |
|---|---|---|
| 093002-08 vs CTRN | <0.0001* | Better luster for 093002-08 |
| 093002-08 vs CTRP | 0.003* | Better luster for 093002-08 |
| CTRP vs CTRN | 0.003* | Better luster for CTRP |

*Significant at 5% (Fisher's LSD test).

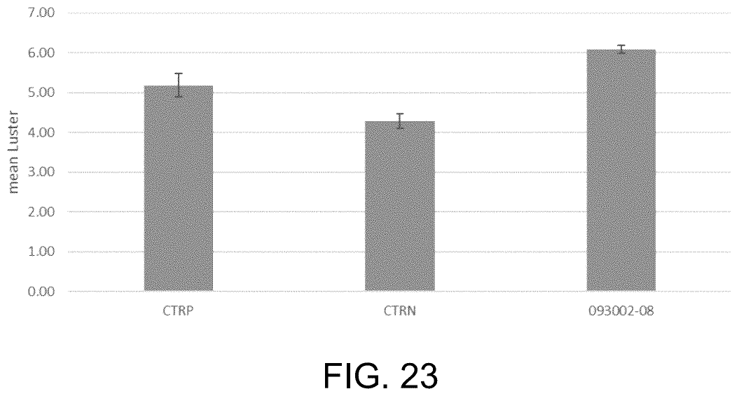

From the statistical comparison presented in Table 216 and FIG. 23, it was possible to verify that the treatment 093002-08 presented a statistically greater luster compared to CTRP and CTRN. The treatment CTRP presented a statistically greater luster compared to CTRN. Using the equations disclosed herein, it possible to conclude that the treatment 093002-08 restores 200% of the natural luster of the hair and 1.4 times more luster compared to a sebum.
Frizz—Table 17 presents the results observed and descriptive statistics by treatment for analysis of frizz.

TABLE 17
Mean and Standard Error by treatment evaluated

| | T0 | | T24 | |
|---|---|---|---|---|
| Statistics | 093002-08 | CTR | 093002-08 | CTR |
| Mean | 1.70 | 71.39 | 2.04 | 80.10 |
| St. Error | 0.17 | 5.66 | 0.28 | 3.57 |
| 95% CI | [1.27 ± 2.13] | [56.85 ± 85.93] | [1.32 ± 2.76] | [70.93 ± 89.27] |
| St. Dev. | 0.41 | 13.85 | 0.68 | 8.74 |
| Median | 1.77 | 69.48 | 2.00 | 80.29 |
| Minimum | 0.99 | 57.16 | 1.01 | 69.83 |
| Maximum | 2.25 | 91.72 | 3.14 | 90.58 |

| | | |
|---|---|---|
| p-value | <0.001* | <0.001* |

*Significant at the 5% level (Student's t test)

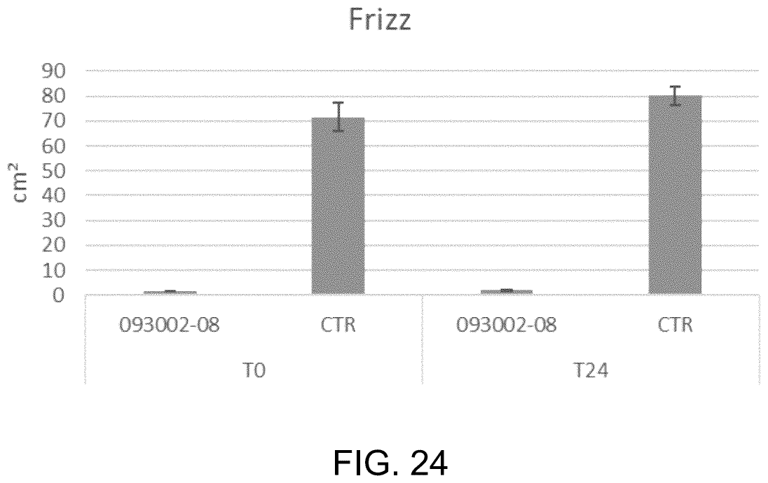

From the statistical comparison presented in Table 17 and FIG. 24, it was possible to verify that the mean for treatment 092003-08 was significantly lower compared to Control for T0 and T24. No statistically significant difference on the mean was observed between time-points T0 and T24 for 093002-08.
Conclusion—The treatment complete pre-wash scalp oil composition presented 93% Breakage reduction and 15 times less breakage compared to the non-conditioning shampoo. Treatment with the complete pre-wash scalp oil composition presented hair surface protection compared to the non-conditioning shampoo due to surface oil deposition. Therefore, it was possible to observe 94% damage repair and 19 times less damage compared to the non-conditioning shampoo. Treatment with the complete pre-wash scalp oil composition presented 57% split ends reduction and 2 times fewer split ends compared to the non-conditioning shampoo. Treatment with the complete pre-wash scalp oil composition presented 12% more strength compared to the non-conditioning shampoo. Treatment with the complete pre-wash scalp oil composition presented 100% luster (shine) restoration when compared to the positive and negative control groups. Treatment with the complete pre-wash scalp oil composition presented 98% frizz reduction and 97% frizz control and 41 times less frizz compared to the non-conditioning shampoo. Treatment with the complete pre-wash scalp oil composition presented 82% volume reduction and 84% volume control and 6 times less volume compared to the non-conditioning shampoo. Treatment with the complete pre-wash scalp oil composition presented 30% more combability on the wet hair compared to the non-conditioning shampoo. Treatment with the complete pre-wash scalp oil composition presented 72% more combability and was 4 times easier to comb on the dry hair compared to the non-conditioning shampoo. Treatment with the complete pre-wash scalp oil composition presented 8% hydrophobicity restore when compared to the positive and negative control. Treatment with the complete pre-wash scalp oil composition presented Hair restoration.

### Example 5—Evaluation of Thermal Protection via Differential Scanning Calorimetry (DSC) with a Nourishing Shine Drop Composition

Objective—The objective of the present study was to evaluate the thermal protection provided after the application of the nourishing shine drop composition.
Methodology—Two groups containing 1 natural type II swatch were selected by treatment, as follows: Control Group (CTR; non-conditioning shampoo. LESS 20%); and Group TRT1: nourishing shine drop composition—(097009-01). Before applying the treatments, all hair swatches were washed with non-conditioning shampoo to remove residues: 4 ml/g of hair was applied in the hair swatches and spread on wet hair. The hair swatch was massaged 6 times from root to tip and then, rinsed in running water (4±1 L/min and 35±2° C.) for 30 seconds. The swatches from the control group were left to dry for a period of 24 hours in a controlled environment. The remaining hair swatch received the application of treatment line as follows: After application of the control shampoo, excess water was removed and then 0.2 ml/g of hair was applied to the swatches from root to tip. For better product distribution, the swatch was massaged 6 times from root to tip. After applying the treatments, the swatches were damaged as follows: Drying the hair swatches using a blow drier at 200° C.; and after drying, the swatches where flat ironed 20 times. After damaged, the swatches were sent to an external partner and five DSC (TA Instruments) measurements were taken for each treatment: 5 mg of hair cut in pieces of 1 to 2 mm were inserted in Aluminum crucibles and the hair samples were dehydrated at 150° C. for 30 minutes. The measurements were done from 25 to 280° C. with a 50 ml/min nitrogen flow rate. For each curve the enthalpy of Keratin denaturation was measured (FIG. 37) and the mean value of enthalpy for the five measurements statistically compared. The mean values of thermal protection of the evaluated treatments were compared using the student t test model followed considering a confidence interval of 95%.

Table 18 presents the statistical results observed in the comparison between Nourishing Shine Drops and non-conditioning shampoo.

TABLE 18
Descriptive statistics and result of comparison
between treatment and control.

| Statistics | 097009-01 | CTR |
|---|---|---|
| | 8.4 | 7.5 |
| | 8.4 | 7.8 |
| | 8.0 | 8.1 |
| | 8.7 | 8.5 |
| | 9.9 | 7.8 |
| Mean | 8.7 | 7.9 |
| St. Error | 0.3 | 0.2 |
| 95% CI | [7.8; 9.6] | [7.5; 8.4] |
| St. Deviation | 0.7 | 0.4 |
| Median | 8.4 | 7.8 |
| Minimum | 8.0 | 7.5 |
| Maximum | 9.9 | 8.5 |

| | |
|---|---|
| p-value | 0.089 |

*Significant at 5% (Student's t test)

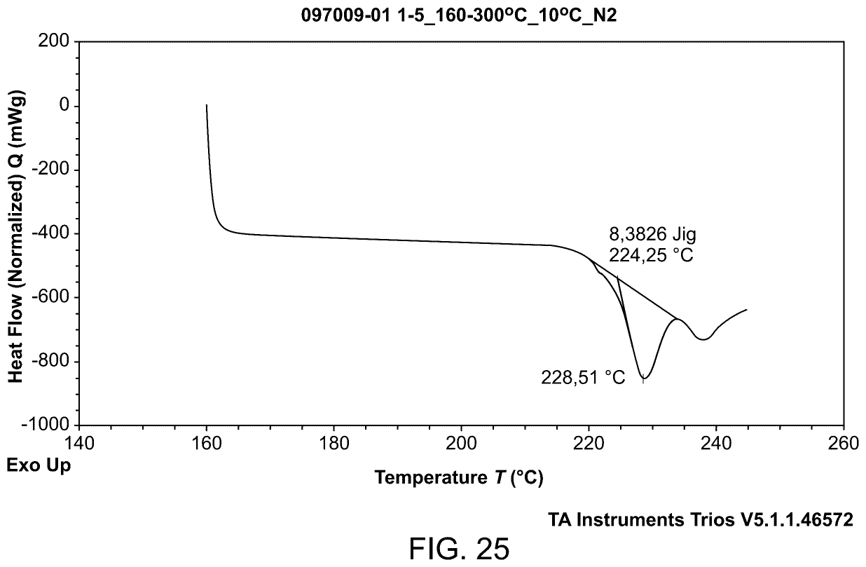

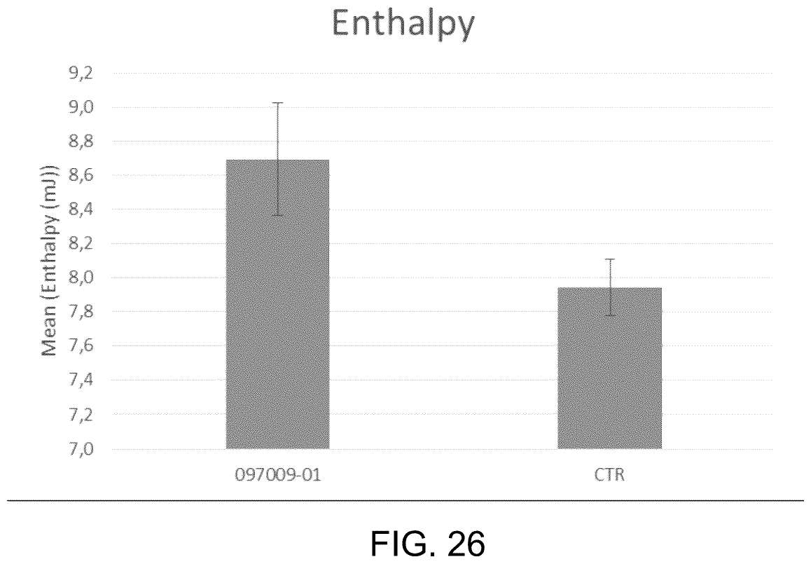

According to the data presented in table 18, FIG. 25, FIG. 26, and the statistical comparison performed, at a 95% confidence interval no statistically significant difference was observed in keratin denaturation enthalpy for treatment 097009-01 compared to non-conditioning shampoo. However, at a 90% confidence interval the treatment 097009-01 presented a higher keratin denaturation enthalpy compared to the non-conditioning shampoo. Therefore, presenting thermal protection.

Conclusion—the nourishing shine drop composition presented thermal protection compared to the non-conditioning shampoo.

### Example 6—Evaluation of Damage Restoration with a Nourishing Shine Drop Composition

Objective—The objective of the present study was to evaluate the restoration provided after the application of a nourishing shine drop composition.
Methodology—Two groups containing 1 type II bleached swatches were separated by treatment as described below: Control Group (CTR; non-conditioning shampoo LESS 20%) and Group TRT1: nourishing shine drop composition—(097009-01). Before applying the treatments, all hair swatches were washed with non-conditioning shampoo to remove residues: 4 ml/g of hair was applied in the hair swatches and spread on wet hair. The hair swatch was massaged 6 times from the root to tip and then, rinsed in running water (4±1 L/min and 37±2° C.) for 30 seconds. The swatches from the control group were left to dry for a period of 24 hours in a controlled environment. The remaining hair swatch received the application of treatment line according to description below: After application of the control shampoo, excess water was removed and then 0.2 ml/g of hair was applied to the swatches from root to tip. For better product distribution, the swatch was massaged 6 times from the root to tip. After application and drying, the swatches were sent to a third-party institute to be evaluated. The images were performed in a scanning electron microscope with a field emission electron gun, FEG-MEV, Zeiss model Supra 35. Obtaining these images aims to analyze the surface profile of hair fibers after the treatments previously described. For this, six fibers of the treated swatch were randomly removed and placed in aluminum stubs with graphite tape so that electrons could flow along the fiber without damaging it. The fibers are scanned by a microscope and six images (one of each fiber) with 2000× magnification amplitude are taken, highlighting the profile presented in the scan. Hair is a natural keratin fiber, a protein composed of a high degree of sulfur content present in the cysteine compounds. The mechanical properties observed in the hair depend, mainly, on its geometric structure and are very important in the evaluation of elasticity, softness and smoothness, combability, shine and so on. The evaluation of these properties by different methodologies can provide a better understanding of the real action of products on the hair to provide better marketing communications for these products. In the present study, studies were done to evaluate the treatments Nourishing Shine Drops compared to a non-conditioning shampoo: Surface analysis to evaluate damage repair via SEM. The mean damage values of the evaluated treatment were compared using t test considering a confidence interval of 95%. The equations below can be used, always a difference is observed, to estimate the percentage of reduction and the number of times the product is better compared to 20% SLES. TRT and SLES stands for the mean value of the treatment and 20% S LES, respectively, in each methodology used:

$$\begin{matrix}\% = ( 1 - \frac{treatment}{control} ) \times 100 & ( Eq . 1 ) \\ Number of times = ( \frac{treatment}{control} ) - 1 & ( Eq . 2 )\end{matrix}$$

Results—Table 9 presents the results observed and descriptive statistics by treatment for analysis of damage of fiber the product 097009-01.

TABLE 19
Mean and Standard Error by treatment evaluated

| Statistics | 097009-01 | CTR |
|---|---|---|
| | 93.7 | 170.9 |
| | 145.8 | 215.7 |
| | 34.7 | 310.3 |
| | 78.9 | 165.7 |
| | 110.4 | 147.2 |
| | 47.1 | 104.6 |
| Mean | 85.1 | 185.7 |
| St. Error | 16.8 | 28.9 |
| 95% CI | [42; 128.2] | [111.4; 260.1] |
| St. Deviation | 41.0 | 70.9 |
| Median | 86.3 | 168.3 |
| Minimum | 34.7 | 104.6 |
| Maximum | 145.8 | 310.3 |

| | |
|---|---|
| p-value | 0.017 |

*significant at 5% (Student's t test).

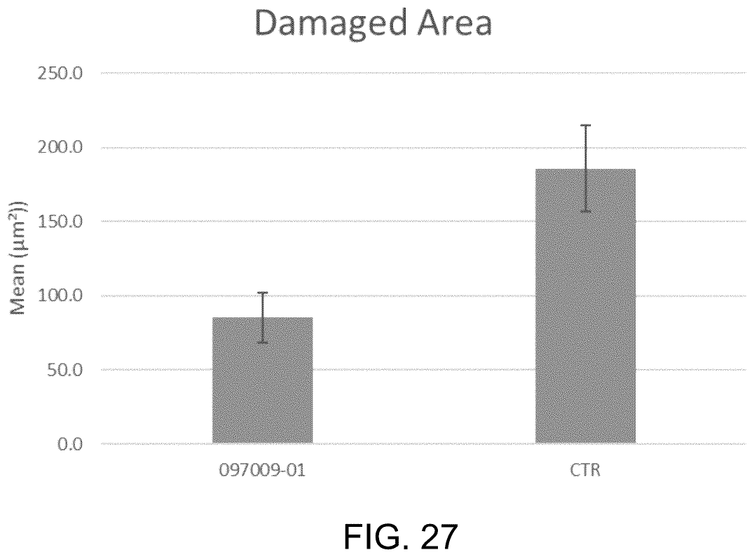

From the data presented in Table 19, FIG. 27 and the statistical analyses performed, it was possible to observe statistically significant lower damaged area was observed for treatment 097009-01 in comparison with CTR.
Conclusion—Treatment with a nourishing shine drop composition presented hair surface protection compared to the non-conditioning shampoo. Therefore, it was possible to observe 54% damage repair and 2 times less damage compared to the non-conditioning shampoo.

### Example 7—Evaluation of Frizz and Volume with a Nourishing Shine Drop Composition

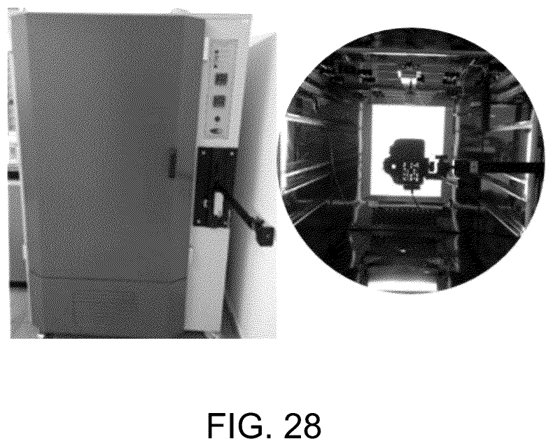

Objective—The objective of the present study was to evaluate the reduction and control of frizz and volume provided after the application of a nourishing shine drop composition.
Methodology—Six natural hair swatches, Type III of 3 grams and 20 cm were separated for each group: Control Group (CTR; Non-conditioning shampoo LESS 20%) and Group TRT1: nourishing shine drop composition—(097009-01). Before applying the treatments, all hair swatches were washed with non-conditioning shampoo to remove any residue the swatches might have: 0.4 grams of 20% SLES per gram of hair was applied on the hair swatches and spread on wet hair. The hair swatch was massaged 6 times from the roots to tip and then, rinsed in running water (5±1 L/min and 35±2° C.) for 30 seconds. Six hair swatches with non-conditioning shampoo were randomly selected and inserted in a humidity and temperature control chamber (55±5% RH; 22±2° C.) where they remained for 24 hours. After that, the hair swatches were photographed, and the images stored for further analysis of frizz and volume (T0). The remaining hair swatches were treated as follow: After application of the control shampoo, excess water was removed and then 0.2 ml/g of hair was applied to the swatches from root to tip. For better product distribution, the swatch was massaged 6 times from the from root to tip. After applying the treatments, the hair swatches were inserted in a humidity and temperature control chamber (55±5% RH; 22±2° C.) where they remained for 24 hours. After that, the hair swatches were photographed, and the images stored for further analysis of frizz and volume (T0). After taken the photographs of T0 (24 hour in low humidity), the humidity was increased to 85±5% RH and the swatches remained in that environment for further 24 hours. After that, the swatches were photographed, and the images stored for further analysis of frizz and volume (T24). FIG. 28 presents the humidity and temperature control chamber used in the test and the system for images acquisition. FIG. 3 presents the images treated in the software ImageJ® using an internally developed macro where three different regions can be assessed: body, frizz and volume.
The mean values of frizz and volume of the evaluated treatments were compared using ANOVA model considering a 95% confidence interval. Equation 1 calculates the % of frizz and volume reduction whenever the treatment differs from the control. Equation 2 calculates how many times less frizz and volume the treatment provides compared to the control whenever the treatment differs from the control. The % reduction and number of times values will be presented at the conclusion of this report.

$$\begin{matrix}\% reduction ( control ) = ( 1 - \frac{TRT}{CTR} ) \times 100 & Eq . 1 \\ Number of times = {( \frac{TRT}{CTR} )}^{-} & Eq . 2\end{matrix}$$

Results—Frizz—Table 19 presents the statistical results observed in the comparison among the treatments and non-conditioning shampoo after In low humidity (55±5% and 20° C.±2° C.).

TABLE 19
Descriptive statistics and results of the
comparison between treatment and control.

T0

| Statistics | CTR | 097009-01 |
|---|---|---|
| Mean | 77.11 | 3.28 |
| Standard error | 3.54 | 0.49 |
| 95% CI | [68.01 ± 86.21] | [2.03 ± 4.53] |
| Standard Deviation | 8.67 | 1.19 |
| Median | 74.72 | 3.27 |
| Minimum | 67.93 | 1.47 |
| Maximum | 91.89 | 5.09 |

| | |
|---|---|
| p-value | <0.001* |

*Significant at 5% (Tukey's test).

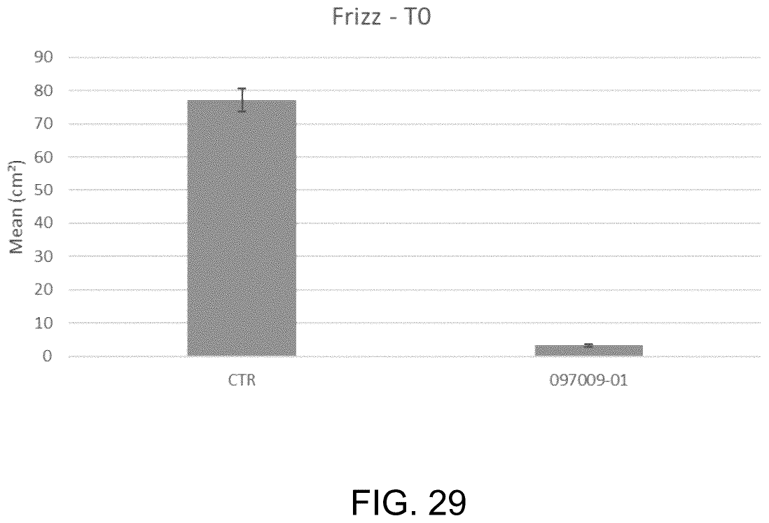

According to the data presented in table 19, FIG. 29 and the statistical comparison performed, it was possible to observe that the frizz for treatment 097009-01 was significantly lower compared to Control at time-point T0.

Table 20 present the statistical results observed in the comparison among the treatments and non-conditioning shampoo after In low humidity (85±5% and 20° C.±2° C.).

TABLE 20
Descriptive statistics and result of comparison
between treatment and control.

T24

| Statistics | CTR | 094907-01 |
|---|---|---|
| Mean | 95.20 | 4.02 |
| Standard error | 4.42 | 0.58 |
| 95% CI | [83.84 ± 106.56] | [2.54 ± 5.5] |
| Standard Deviation | 10.83 | 1.41 |
| Median | 94.39 | 3.81 |
| Minimum | 82.27 | 2.28 |
| Maximum | 114.97 | 5.72 |

| | |
|---|---|
| p-value | <0.001* |

*Significant at 5% (paired Student t test).

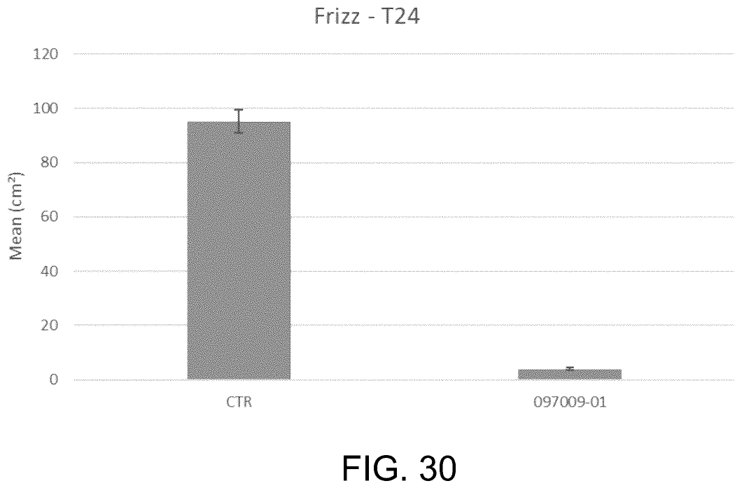

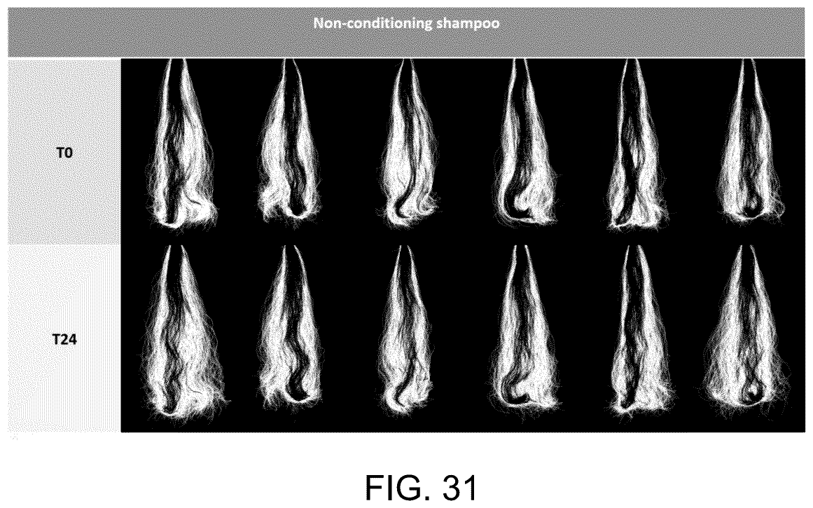

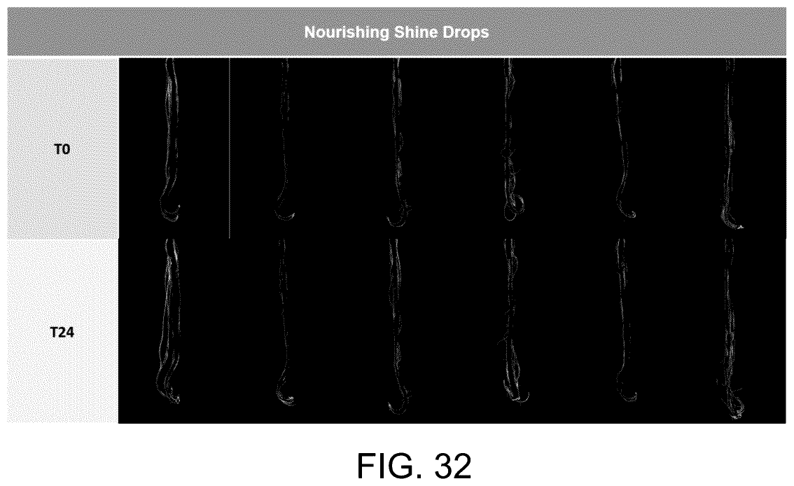

According to the data presented in table 20, FIG. 30 and the statistical comparison performed, it was possible to observe that the frizz for treatment 097009-01 was significantly lower compared to Control at time-point T24. FIGS. 31 and 32 illustrate this effect.

Table 21 present the statistical results observed in the comparison among the treatments and non-conditioning shampoo after in low humidity (85±5% and 20° C.±2° C.).

TABLE 21
Descriptive statistics and result of comparison
between treatment and control.

T24

| Statistics | CTR | 097009-01 |
|---|---|---|
| Mean | 150.93 | 25.13 |
| Standard error | 6.12 | 1.76 |
| 95% CI | [135.19 ± 166.67] | [20.61 ± 29.65] |
| Standard Deviation | 15.00 | 4.31 |
| Median | 151.68 | 24.60 |
| Minimum | 125.81 | 19.96 |
| Maximum | 172.67 | 32.00 |
| p-value | <0.001* | |

*Significant at 5% (Student's t test).

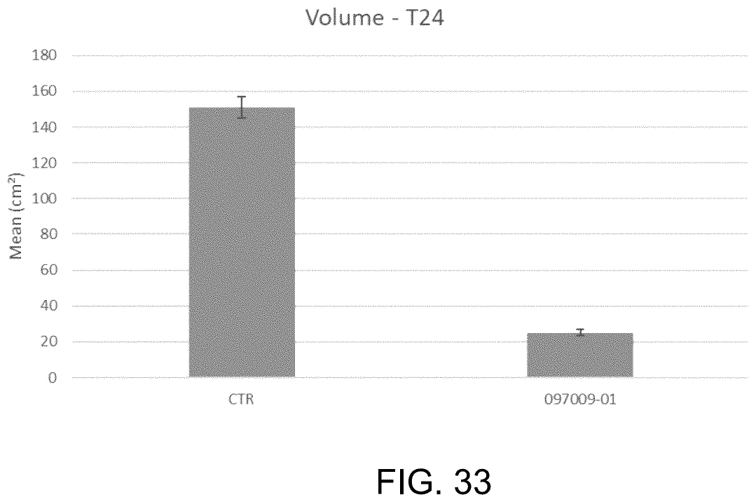

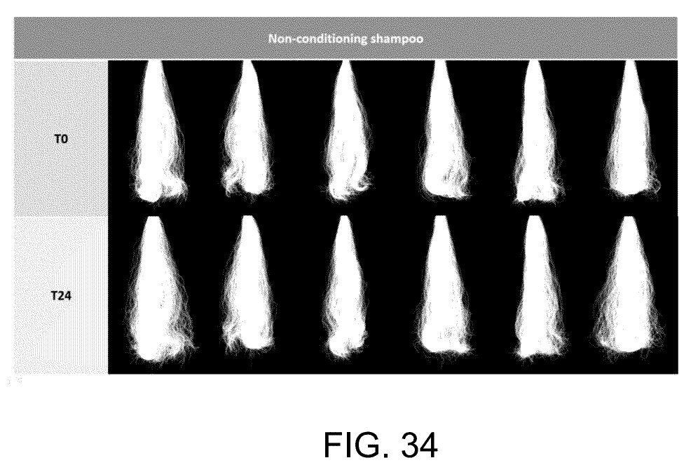

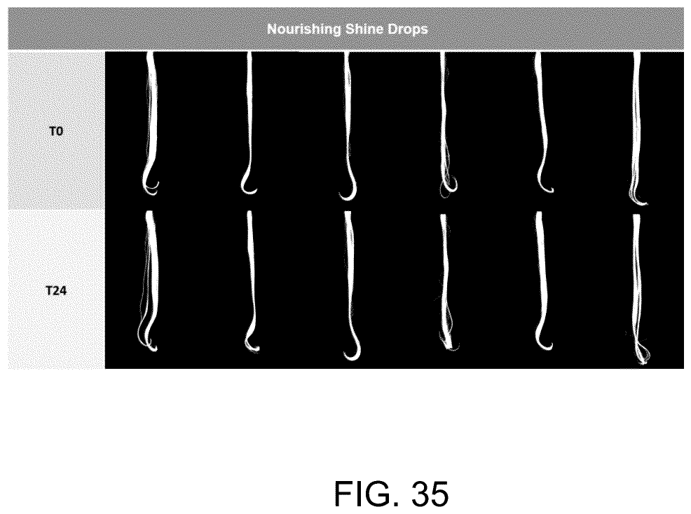

According to the data presented in table 21, FIG. 33, FIG. 34 and FIG. 35, the statistical comparison performed, it was possible to observe that the volume for treatment 097009-01 was significantly lower compared to Control at time-point T24.

Conclusion—The nourishing shine drop composition treatment presented 96% of frizz reduction and 24 times less frizz compared to the non-conditioning shampoo. The nourishing shine drop composition treatment presented 83% of frizz control and 6 times less frizz after 24 hours of high humidity exposure.

### Example 8—Evaluation of UV Protection with a Nourishing Shine Drop Composition

Objective—The objective of the present study was to evaluate the UV protection provided after the application of a nourishing shine drop composition
Methodology—Three Brazilian natural hair swatches type II of 20 cm and 3 grams were prepared and separated into three different groups: CTRL: without exposition to UV radiation; CTRL negative: 96 hour of UV radiation exposure; and TRAT1: nourishing shine drop composition (097009-01) application followed by 96 hours of UV radiation exposure. Before applying the treatments, all hair swatches were washed with non-conditioning shampoo to remove residues: 0.4 ml/g of hair was applied in the hair swatches and spread on wet hair. The hair swatch was massaged 6 times from root to tip and then, rinsed in running water (4±1 L/min and 35±2° C.) for 30 seconds. The swatches from the control group were left to dry for a period of 24 hours in a controlled environment. The remaining hairs swatches received the application of treatment as follows: After application of the control shampoo, excess water was removed and then 0.2 ml/g of hair was applied to the swatches from root to tip. For better product distribution, the swatch was massaged 6 times from root to tip. After drying, the swatches were exposed to UV ray and then evaluated. After the UV radiation exposure, the hair swatches were analyzed by a spectrophotometer Fluorolog-Jobin Yvon Horibe FL3-12 with a monochromator Xenon lamp to evaluate the tryptophan (Trp) content of the hair. Three measurements were done by hair swatch and the mean value compared statistically among the groups. The closer the tryptophan content of the treatment compared to CTRL the higher the UV protection. Table 22 and FIG. 15 show, respectively, the equipment configuration and the spectral profile of the lamp.

TABLE 22
Atlas Weather-Ometer specifications

| | |
|---|---|
| Radiation source | 6500 W Xenon lamp |
| Aging cycle | Without insulation and rain exposure |
| Control | Irradiance of 0.35 W/m² at 340 nm |
| Total density energy | 2641 mW/cm² |

The mean values of UV protection of the evaluated treatments were compared using the student t test model followed considering a confidence interval of 95%. UV protection of the treatments whenever a difference is observed when compared to the negative (CTRLN) and positive (CTRLP) control was calculated using the following equation:

$$[ 1 - ( \frac{( CTRLP - CTRLN ) - ( TRAT - CTRLN )}{( CTRLP - CTRLN )} ) ] * 100$$

Table 34 shows the results of the six measurements of tryptophan emission at 325 nm (maximum emission.

TABLE 23
Tryptophan emission at 325 nm by treatment.

| Measurement | CTRL | CTRL negative | TRAT1 |
|---|---|---|---|
| 1 | 44.063 | 35.410 | 65.959 |
| 2 | 43.737 | 35.974 | 67.302 |
| 3 | 43.240 | 37.935 | 61.698 |
| 4 | 72.290 | 30.287 | 50.705 |
| 5 | 70.774 | 29.740 | 50.968 |
| 6 | 70.241 | 26.642 | 52.621 |
| Mean tryptophan value | 57.391 | 32.665 | 58.209 |
| STD deviation | 15.037 | 4.399 | 7.680 |

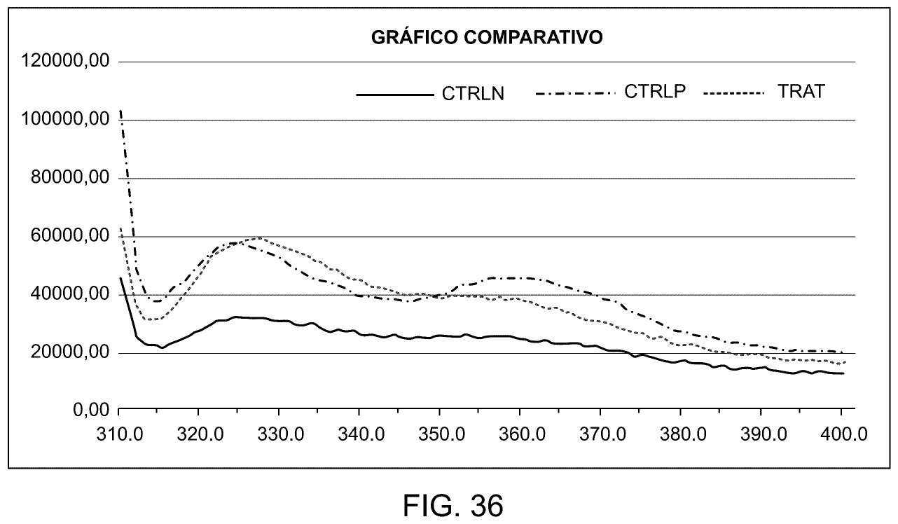

According to the results presented in table 23 and FIG. 36, the statistical analysis performed it was possible to observe that a statistically significant difference was observed between hair swatches of the treatment with 097009-01 and negative control (exposed swatches without radiation protection). There was no statistical difference between the hair swatches that received the application of the treatment 097009-01 irradiated and the positive control swatch (without exposure to radiation). Using the equation above, it was possible to observe the following percentage of protection. Treatment with the nourishing shine drop composition (097009-01) presented a UV protection to the hair of approximately, 100%.

All features disclosed in the specification, including the abstract and drawings, and all the steps in any method or process disclosed, may be combined in any combination, except combinations where at least some of such features and/or steps are mutually exclusive. Each feature disclosed in the specification, including abstract and drawings, can be replaced by alternative features serving the same, equivalent or similar purpose, unless expressly stated otherwise. Thus, unless expressly stated otherwise, each feature disclosed is one example only of a generic series of equivalent or similar features. Various modifications of the disclosure, in addition to those described herein, will be apparent to those skilled in the art from the foregoing description. Such modifications are also intended to fall within the scope of the appended claims.

All publications mentioned herein are incorporated by reference in their entirety. Nothing herein is to be construed as an admission that the disclosure is not entitled to antedate such disclosure by virtue of prior disclo

1. A haircare composition comprising a C13-15 alkane, squalane, at least one conditioning agent and at least one antioxidant.

2. The haircare composition of claim 1, wherein the C13-15 alkane is hemisqualane.

3. The haircare composition of claim 1, wherein the squalane comprises between about 80% w/w and about 99% w/w of the haircare composition.

4. The haircare composition of claim 1, wherein the squalane comprises about 91% w/w of the haircare composition.

5. The haircare composition of claim 1, wherein the C13-15 alkane comprises between about 1% w/w and about 10% w/w of the haircare composition.

6. The haircare composition of claim 1, wherein the C13-15 alkane comprises about 5% w/w of the haircare composition.

7. The haircare composition of claim 1, wherein the conditioning agent is selected from the group consisting oryza sativa (rice) bran extract, helianthus annuus (sunflower) extract, bisabolol, ocimum basilicum hairy root culture extract, helianthus annuus (sunflower) seed oil, cocos nucifera (coconut) oil, curcuma longa root extract, melia azadirachta flower extract, melia azadirachta leaf extract, melia azadirachta bark extract, ocimum sanctum leaf extract, corallina officinalis extract, ocimum basilicum flower/leaf extract, eclipta prostrata extract, melia azadirachta extract, moringa oleifera seed oil, and any combination thereof.

8. The haircare composition of claim 1, wherein the antioxidant is selected from the group consisting of rosmarinus officinalis (rosemary) leaf extract, tocopherol, and a combination thereof.

9. The haircare composition of claim 1, further comprising an aromatic component.

10. The haircare composition of claim 1, wherein the aromatic component is selected from the group consisting of fragrance, citral, limonene, geraniol, linalool, citrus reticulata (tangerine) peel oil, citral, linalool, limonene, jasminum officinale (jasmine) oil, benzyl benzoate, linalool, and any combination thereof.

11. The haircare composition of claim 1, wherein the haircare composition is substantially free from silicones.

12. The haircare composition of claim 1, wherein the haircare composition is free from silicones.

13. A method of reducing hair breakage, hair damage, hair fall, or a combination thereof, comprising administering the haircare composition of claim 1 to the scalp.

14. The method of claim 13, further comprising massaging the haircare composition of claim 1 into the scalp, leaving on for about 10 to about 15 minutes, followed by washing the scalp and hair with shampoo.

15. The haircare composition of claim 1, further comprising a humectant and a film former, and at least one conditioning agent.

16.-46. (canceled)

47. A method of enhancing hair definition, reducing frizz, reducing the appearance of split ends, or any combination thereof, comprising applying the haircare composition of claim 15 to the hair.

48. (canceled)

49. The haircare composition of claim 1, further comprising at least one emollient.

50.-70. (canceled)

71. The haircare composition of claim 1, further comprising a humectant.

72.-92. (canceled)

93. A method of minimizing split ends, improving hair luster, or a combination thereof, comprising applying the haircare composition of claim 71 to the hair.

94. (canceled)

95. A method of protecting against thermal damage, UV damage, or a combination thereof, comprising applying the haircare composition of claim 71 to the hair.

96. (canceled)
